(19) United States
(12) Patent Application Publication
(10) Pub. No.: US 2026/0345504 A1
(43) Pub. Date: Nov. 5, 2026

(54) METHOD OF USING NEURAL STEM CELL-DERIVED EXTRACELLULAR VESICLES FOR TREATING ALZHEIMER'S DISEASE

(71) Applicant: Tongji University, Shanghai (CN)

(72) Inventors: Jialin Charles Zheng, Shanghai (CN); Xiaohuan Xia, Shanghai (CN)

(21) Appl. No.: 19/486,905

(22) PCT Filed: May 22, 2024
(86) PCT No.: PCT/CN2024/094667

§ 371 Date: Nov. 21, 2025

(30) Foreign Application Priority Data
May 22, 2023 (WO) PCT/CN2023/095483

(51) Int. Cl. A61K 9/50; A61K 35/28; A61K 35/30; A61P 25/28

(52) U.S. Cl. CPC A61K 9/5068; A61K 35/28; A61K 35/30; A61P 25/28

(57) ABSTRACT

The present invention relates to a composition and a method for treating Alzheimer's disease in a patient in need thereof. The method comprises administration to the patient of the composition comprising a therapeutically effective amount of extracellular vesicles, wherein the extracellular vesicles are isolated from culture medium of cells. In some embodiments, the extracellular vesicles comprise one or more of following miRNAs: let-7, miR-9, miR-21, miR-34a, and miR-10b.

### CROSS-REFERENCE TO RELATED APPLICATIONS

This application claims priority of PCT Application No. PCT/CN2023/095483, filed on May 22, 2023, the contents of which are incorporated herein by reference in their entirety.

### BACKGROUND OF THE INVENTION

Dementia describes an intra-individual pattern of decline in memory and thinking impairing at least two domains of cognition (McKhann et al., Alzheimers Dement., 7 (2011), pp. 263-269). Alzheimer's disease (AD) is the most common neurodegenerative disorder and the No. 1 cause of dementia in elderly (Leng, F. et al., Nat Rev Neurol. 17(3):157-172 (2021)).

Dementia due to AD is associated with the onset of significant and progressive disability throughout the disease course, with death an inevitable outcome generally occurring within 5-12 years of symptom onset (Vermunt et al., Alzheimers Dement., 15 (2019), pp. 888-898). The burden on caregivers and the public health sector is enormous.

There is a dire need for disease-modifying therapies that may prevent or slow the rate of disease progression, but unfortunately none are currently available.

### SUMMARY OF THE INVENTION

In one aspect, the present invention provides a method for treating Alzheimer's disease in a patient in need thereof. The method includes administration to the patient of a composition comprising a therapeutically effective amount of extracellular vesicles.

In some embodiments, the extracellular vesicles are isolated from culture medium of cells.

In some embodiments, the cells are neural stem cells (NSCs), mesenchymal stem cells (MSCs), or fibroblast derived induced NSCs (iNSCs).

In some embodiments, the extracellular vesicles include one or more of following miRNAs: let-7, miR-9, miR-21, miR-34a, and miR-10b.

In some embodiments, the extracellular vesicles include let-7, miR-21, and miR-10b.

In some embodiments, the administration is once or more than once a day.

In some embodiments, the administration is at least once every three days.

In some embodiments, the administration is at least for a month.

In some embodiments, the administration is via oral, intravenous injectable, intramuscular injectable, intraperitoneal, intranasal, rectal, or sublingual route.

In some embodiments, the administration of the composition is by a dose in the range of 2-2,000 mg, 2-500 mg, 2-200 mg, 2-150 mg, 5-100 mg, or 5-50 mg.

In some embodiments, the dose is administered in divided doses or a single dose.

In some embodiments, the composition down-regulates levels of Aβ1-42 in prefrontal cortex (PFC) and hippocampus.

In some embodiments, the composition reduces phosphorylated Tau (pTau) propagation.

In some embodiments, the composition regulates neuroinflammation.

In some embodiments, the composition reduces levels of CD86, iNOS, and IL-1β in the PFC and hippocampus.

In some embodiments, the composition reduces one or more of following gene expression: Il1b, Il12b, Mmp13, and Atp8b4.

In another aspect, the present invention provides a composition comprising a therapeutically effective amount of extracellular vesicles for treating Alzheimer's disease in a patient in need thereof.

In some embodiments, the extracellular vesicles are isolated from culture medium of cells.

In some other embodiments, the cells are neural stem cells (NSCs), mesenchymal stem cells (MSCs), or fibroblast derived induced NSCs (iNSCs).

In some other embodiments, the extracellular vesicles include one or more of following miRNAs: let-7, miR-9, miR-21, miR-34a, and miR-10b.

In some embodiments, the extracellular vesicles include let-7, miR-21, and miR-10b.

In yet another aspect, the present invention provides a method for preparing any one of the compositions disclosed herein.

In still another aspect, the present invention provides use of any one of the compositions disclosed herein for treating Alzheimer's disease in a patient in need thereof.

In some embodiments, the composition is administrated at a dose of 2-2000 mg, 2-500 mg, 2-200 mg, 2-150 mg, 5-100 mg, or 5-50 mg.

In some embodiments, the composition is administrated at least once a day or more times a day.

In some embodiments, the administration is by oral, intravenous injectable, intramuscular injectable, intraperitoneal, intranasal, rectal, or sublingual route.

These and other features, aspects, and advantages of the present invention will become better understood with reference to the following description and appended claims.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1B are characterization of NSCs and iNSCs. (FIG. 1A) Representative confocal microscopy images of Sox2, Nestin, and Ki67 immunoreactivities in the neurospheres generated by NSCs and iNSCs. (FIG. 1B) Representative confocal microscopy images of Sox2, Nestin, and Ki67 immunoreactivities in NSCs and iNSCs in adherent cell culture. Scale bar: 50 μm.

FIG. 2A-FIG. 2E are further characterization of NSC-EVs and iNSC-EVs. (FIG. 2A) Representative blots of positive EV markers Flotillin1, Flotillin2, and CD9 in NSC- and iNSC-EVs. (FIG. 2B) Representative blots of negative EV markers APOA1 and APOA2 in EVs and their parent cells. (FIG. 2C) NTA analysis of NSC- and iNSC-EVs. (FIG. 2D) SEM of NSCs and iNSCs. (FIG. 2E) TEM characterization of the morphology of NSC- and iNSC-EVs. Scale bar: 2 μm (FIG. 2D), and 200 nm (FIG. 2E).

FIG. 3 is in vivo imaging of Dil-labeled NSC-EVs and iNSC-EVs after intravenous administration. Mice were injected intravenously with Dil-labeled EVs. The distribution of Dil-labeled EVs was observed by in vivo fluorescence imaging, showing distributed Dil-labeled EVs throughout the whole bodies of mice from 5 min to 360 min after EVs injection. PBS-injected mice were used as negative controls.

FIG. 4 shows fluorescence images of mouse brain tissues after intravenous administration of Dil-labeled NSC-EVs and iNSC-EVs. Mouse brains were dissected out at different time points after intravenous injection of Dil-labeled EVs. Brains of PBS-injected mice were used as negative controls.

FIG. 5A-FIG. 5C show the uptake of NSC-EVs and iNSC-EVs by cortical cells after intravenous administration. Mice were injected intravenously with Dil-labeled EVs. Brain tissues were collected 5 min after EVs injection. (FIG. 5A) Representative confocal microscopy images of Tuj1, CD9, and Dil immunoreactivities in the cortex. (FIG. 5B) Representative confocal microscopy images of GFAP, CD9, and Dil immunoreactivities in the cortex. (FIG. 5C) Representative confocal microscopy images of Iba1, CD9, and Dil immunoreactivities in the cortex. Images at the bottom panels were high-magnification images of the corresponding small box area from the top panels in each group. Arrows indicate overlapping signals. Scale bar: 20 μm.

FIG. 6A-FIG. 6C show the uptake of NSC-EVs and iNSC-EVs by hippocampal cells after intravenous administration. Mice were injected intravenously with Dil-labeled EVs. Brain tissues were collected 5 min after EVs injection. (FIG. 6A) Representative confocal microscopy images of Tuj1, CD9, and Dil immunoreactivities in the hippocampus. (FIG. 6B) Representative confocal microscopy images of GFAP, CD9, and Dil immunoreactivities in the hippocampus. (FIG. 6C) Representative confocal microscopy images of Iba1, CD9, and Dil immunoreactivities in the hippocampus. Images at the bottom panels were high-magnification images of the corresponding small box area from the top panels in each group. Arrows indicate overlapping signals. Scale bar: 20 μm.

FIG. 7 shows intravenous administration of NSC-EVs and iNSC-EVs does not alter motor functions of 5×FAD mice in Morris water maze. The total swimming distance and average swimming speed of mice in Morris water maze (n=9). The statistical difference among groups was assessed with the parametric one-way ANOVA with post-hoc Bonferroni test.

FIG. 8 shows intravenous administration of NSC-EVs and iNSC-EVs does not alter motor ability of 5×FAD mice in Y maze test. The total travel distance and average speed of mice in Y maze test (n=9). The statistical difference among groups was assessed with the parametric one-way ANOVA with post-hoc Bonferroni test.

FIG. 9A-FIG. 9D show intravenous administration of NSC-EVs and iNSC-EVs mitigates Aβ plaque deposition in the hippocampi of 5×FAD mice. (FIG. 9A) Representative confocal microscopy images of Aβ immunoreactivity in the hippocampus at ×20 magnification. (FIG. 9B FIG. 9D) Aβ plaque burden (FIG. 9B), density (FIG. 9C), and average size (FIG. 9D) were quantified using ImageJ (n=4). Scale bar: 200 μm. Error bars denote s.d. *, **, ***, **** denote p<0.05, p<0.01, p<0.001, and p<0.0001, respectively. The statistical difference among groups was assessed with the parametric one-way ANOVA with post-hoc Bonferroni test.

FIG. 10 shows intravenous administration of NSC-EVs and iNSC-EVs reduces Aβ accumulation in the hippocampi of 5×FAD mice. The levels of Aβ₁₋₄₂ in the hippocampus were determined by ELISA assay (n=6). Error bars denote s.d. * and ** denote p<0.05 and p<0.01, respectively. The statistical difference among groups was assessed with the parametric one-way ANOVA with post-hoc Bonferroni test.

FIG. 11 shows intravenous administration of NSC-EVs and iNSC-EVs alleviates pTau propagation in the hippocampi of 5×FAD mice. Representative confocal microscopy images of pTau immunoreactivity in the hippocampus at ×20 magnification. The intensity of pTau signal was quantified using ImageJ (n=6). Scale bar: 200 μm. Error bars denote s.d. **** denotes p<0.0001. The statistical difference among groups was assessed with the parametric one-way ANOVA with post-hoc Bonferroni test.

FIG. 12A-FIG. 12D show intravenous administration of NSC-EVs and iNSC-EVs restores dendritic length and spine density in the hippocampi of 5×FAD mice. (FIG. 12A) Representative microscopy images of Golgi-Cox staining of hippocampal tissue samples at ×40 magnification. (FIG. 12B) Enlarged images demonstrating dendrites and spines. (FIG. 12C) Quantification of dendritic length of neurons in hippocampal tissue samples (n=9). (FIG. 12D) Quantification of spine density in hippocampal tissue samples (n=10). Error bars denote s.d. *, **, ***, **** denotes p<0.05, p<0.01, p<0.001, p<0.0001, respectively. The statistical difference among groups was assessed with the parametric one-way ANOVA with post-hoc Bonferroni test.

FIG. 13A-FIG. 13F show intravenous administration of NSC-EVs and iNSC-EVs enhance cognitive performance of 5×FAD mice in Morris water maze. (FIG. 13A) Swim paths of mice while the platform was removed (memory phase); (FIG. 13B) Platform crossing numbers of mice in the target quadrant (n=9). (FIG. 13C) Percentage of swimming distance (s) spent in the target quadrant of the Morris Water Maze (n=9). (FIG. 13D) Percentage of swimming time (s) spent in the target quadrant of the Morris Water Maze (n=9). (FIG. 13E) Average swimming speed (s) of mice in the Morris water maze test (n=9). (FIG. 13F) Average swimming distance (s) of mice in the Morris water maze test (n=9). Error bars denote s.d. n.s. denotes non-significance. *, **, *** denote p<0.05, p<0.01, and p<0.001, respectively. The statistical difference among groups was assessed with the parametric one-way ANOVA with post-hoc Bonferroni test.

FIG. 14A-FIG. 14B show NSC-EVs and iNSC-EVs enhance cognitive performance of 5×FAD mice in fear conditioning test 1 month post intravenous administration. (FIG. 14A) Total freezing time (s) of mice in fear conditioning test. (FIG. 14B) Percentage of freezing time (s) of mice in fear conditioning test (n=9). Error bars denote s.d. n.s. denotes non-significance. **, ***, **** denote p<0.01, p<0.001, and p<0.0001, respectively. The statistical difference among groups was assessed with the parametric one-way ANOVA with post-hoc Bonferroni test.

FIG. 15A-FIG. 15F. NSC-EVs and iNSC-EVs mitigated Aβ plaque deposition in the brains of 5×FAD mice 1 month post intravenous administration. (FIG. 15A) Aβ immunoreactivity in the PFC 1 month post EV administration. (FIG. 15B-FIG. 15C) Aβ plaque density (FIG. 15B) and average size (FIG. 15C) in the PFC were quantified using ImageJ (n=9). (FIG. 15D) Aβ immunoreactivity in the hippocampal tissues 1 month post EV administration. (FIG. 15E-FIG. 15F) Aβ plaque density (FIG. 15E) and average size (FIG. 15F) in the hippocampal tissues were quantified using ImageJ (n=9). Error bars denote s.d. n.s. denotes non-significance. **, ***, **** denote p<0.01, p<0.001, and p<0.0001, respectively. The statistical difference among groups was assessed with the parametric one-way ANOVA with post-hoc Bonferroni test.

FIG. 16A-FIG. 16B. Intravenous administration of NSC-EVs and iNSC-EVs mitigates pTau propagation in the brains of 5×FAD mice. (FIG. 16A) Representative confocal microscopy images of pTau immunoreactivity in the PFC at ×20 magnification. The intensity of pTau signal quantified using ImageJ was provided on the right panel (n=6). (FIG. 16B) Representative confocal microscopy images of pTau immunoreactivity in the hippocampus at ×20 magnification. The intensity of pTau signal quantified using ImageJ was provided on the right panel (n=6). Scale bar: 200 μm. Error bars denote s.d. *** and **** denote p<0.001 and p<0.0001. The statistical difference among groups was assessed with the parametric one-way ANOVA with post-hoc Bonferroni test.

FIG. 17A-FIG. 17H. Intravenous administration of NSC-EVs and iNSC-EVs restores dendritic length and spine density in the brains of 5×FAD mice. (FIG. 17A) Representative microscopy images of Golgi-Cox staining of cortical tissue samples at ×40 magnification. (FIG. 17B) Enlarged images demonstrating dendrites and spines. (FIG. 17C) Quantification of dendritic length of neurons in cortical tissue samples (n=9). (FIG. 17D) Quantification of spine density in cortical I tissue samples (n=10). (FIG. 17E) Representative microscopy images of Golgi-Cox staining of hippocampal tissue samples at ×40 magnification. (FIG. 17F) Enlarged images demonstrating dendrites and spines. (FIG. 17G) Quantification of dendritic length of neurons in hippocampal tissue samples (n=9). (FIG. 17H) Quantification of spine density in hippocampal tissue samples (n=10). Error bars denote s.d. * and *** denotes p<0.05 and p<0.001, respectively. The statistical difference among groups was assessed with the parametric one-way ANOVA with post-hoc Bonferroni test.

FIG. 18A-FIG. 18D. Characterization of NSC-EVs, iNSC-EVs, MSC-EVs, and FB-EVs. (FIG. 18A) Representative blots of positive EV markers Flotillin1, Flotillin2, and CD9 in NSC- and iNSC-EVs. (FIG. 18B) Representative blots of negative EV markers APOA1 and APOA2 in EVs and their parent cells. (FIG. 18C) NTA analysis of EVs. (FIG. 18D) TEM characterization of the morphology of EVs. Scale bar: 200 nm.

FIG. 19A-FIG. 19F. Intravenous administration of stem cell-derived EVs enhance cognitive performance of 5×FAD mice in Morris water maze. (FIG. 19A) Swim paths of mice while the platform was removed (memory phase); (FIG. 19B) Platform crossing numbers of mice in the target quadrant (n=7). (FIG. 19C) Percentage of swimming distance (s) spent in the target quadrant of the Morris Water Maze (n=7). (FIG. 19D) Percentage of swimming time (s) spent in the target quadrant of the Morris Water Maze (n=7). (FIG. 19E) Average swimming speed (s) of mice in the Morris water maze test (n=7). (FIG. 19F) Average swimming distance (s) of mice in the Morris water maze test (n=7). Error bars denote s.d. n.s. denotes non-significance. *, **, ***, **** denote p<0.05, p<0.01, p<0.001, p<0.0001, respectively. The statistical difference among groups was assessed with the parametric one-way ANOVA with post-hoc Bonferroni test.

FIG. 20A-FIG. 20B. Intravenous administration of stem cell-derived EVs alleviated cognitive function of 5×FAD mice in fear conditioning test. (FIG. 20A) Total freezing time (s) of mice in fear conditioning test. (FIG. 20B) Percentage of freezing time (s) of mice in fear conditioning test (n=9). Error bars denote s.d. n.s. denotes non-significance. *, **, ***, **** denote p<0.05, p<0.01, p<0.001, p<0.0001, respectively. The statistical difference among groups was assessed with the parametric one-way ANOVA with post-hoc Bonferroni test.

FIG. 21A-FIG. 21F. Intravenous administration of stem cell-derived EVs mitigates Aβ plaque deposition in the brains of 5×FAD mice. (FIG. 21A) Representative confocal microscopy images of Aβ immunoreactivity in the PFC at ×20 magnification. (FIG. 21B-FIG. 21C) Aβ plaque density (FIG. 21B) and average size (FIG. 21C) in the PFC were quantified using ImageJ (n=10). (FIG. 21D) Representative confocal microscopy images of Aβ immunoreactivity in the PFC at ×20 magnification. (FIG. 21E-FIG. 21F) Aβ plaque density (FIG. 21E) and average size (FIG. 21F) in the hippocampus were quantified using ImageJ (n=10). Scale bar: 200 μm. Error bars denote s.d. *, **, **** denote p<0.05, p<0.01, p<0.0001, respectively. The statistical difference among groups was assessed with the parametric one-way ANOVA with post-hoc Bonferroni test.

FIG. 22A-FIG. 22B. Intravenous administration of stem cell-derived EVs alleviates pTau propagation in the brains of 5×FAD mice. (FIG. 22A) Representative confocal microscopy images of pTau immunoreactivity in the PFC at ×20 magnification. The intensity of pTau signal in the PFC, quantified using ImageJ, was provided in the right panel (n=6). (FIG. 22B) Representative confocal microscopy images of pTau immunoreactivity in the hippocampus at ×20 magnification. The intensity of pTau signal in the hippocampus, quantified using ImageJ, was provided in the right panel (n=6). Scale bar: 200 μm. Error bars denote s.d. *** and **** denote p<0.001 and p<0.0001, respectively. The statistical difference among groups was assessed with the parametric one-way ANOVA with post-hoc Bonferroni test.

FIG. 23A-FIG. 23H. Intravenous administration of Stem cell-derived EVs restores dendritic length and spine density in the brains of 5×FAD mice. (FIG. 23A) Representative microscopy images of Golgi-Cox staining of cortical tissue samples at ×40 magnification. (FIG. 23B) Enlarged images demonstrating dendrites and spines. (FIG. 23C) Quantification of dendritic length of neurons in cortical tissue samples (n=10). (FIG. 23D) Quantification of spine density in hippocampal tissue samples (n=10). (FIG. 23E) Representative microscopy images of Golgi-Cox staining of hippocampal tissue samples at ×40 magnification. (FIG. 23F) Enlarged images demonstrating dendrites and spines. (FIG. 23G) Quantification of dendritic length of neurons in hippocampal tissue samples (n=10). (FIG. 23H) Quantification of spine density in hippocampal tissue samples (n=10). Error bars denote s.d. *, **, ***, **** denotes p<0.05, p<0.01, p<0.001, p<0.0001, respectively. The statistical difference among groups was assessed with the parametric one-way ANOVA with post-hoc Bonferroni test.

FIG. 24A-FIG. 24E. Intravenous administration of NSC- and iNSC-EVs alters gene expression profiles in 5×FAD mouse hippocampal tissues. (FIG. 24A) Heatmap of top 40 DEGs among groups. (FIG. 24B) The top 10 GO terms of DEGs in comparison between NSC-EV-inject mice and PBS controls. (FIG. 24C) The top 10 KEGG pathways of DEGs in comparison between NSC-EV-inject mice and PBS controls. (FIG. 24D) The top 10 GO terms of DEGs in comparison between iNSC-EV-inject mice and PBS controls. (FIG. 24E) The top 10 KEGG pathways of DEGs in comparison between iNSC-EV-inject mice and PBS controls.

FIG. 25A-FIG. 25E. NSC- and iNSC-EVs are internalized mainly by microglia in the brain. Mice were injected intravenously with Dil-labeled EVs. Brain tissues were collected 5 min after EVs injection. (FIG. 25A) Representative confocal microscopy images of Iba1 and Dil immunoreactivities in the cortex and hippocampus. (FIG. 25B) Representative confocal microscopy images of GFAP and Dil immunoreactivities in the cortex and hippocampus. (FIG. 25C) Representative confocal microscopy images of Tuj1 and Dil immunoreactivities in the cortex and hippocampus. (FIG. 25D) Quantitation analysis for the comparison of NSC-EVs intake in microglia, astrocytes, and neurons in the PFC and hippocampus. (FIG. 25E) Quantitation analysis for the comparison of iNSC-EVs intake in microglia, astrocytes, and neurons in the PFC and hippocampus. Images at the right panels were high-magnification images of the corresponding small box area from the left panels in each group. Arrows indicate overlapping signals. Scale bar: 50 μm. Error bars denote s.d. **, ***, and **** denote p<0.01, p<0.001, and p<0.0001, respectively. The statistical difference among groups was assessed with the parametric one-way ANOVA with post-hoc Bonferroni test.

FIG. 26A-FIG. 26H. Intravenous administration of NSC-EVs and iNSC-EVs inhibits microglial activation and enhances microglial Aβ responses in the hippocampi of 5×FAD mice. (FIG. 26A) Representative confocal microscopy images of Iba1 immunoreactivity in the hippocampus at ×20 magnification. (FIG. 26B) Numbers of Iba1⁺ cells in each group (n=6). (FIG. 26C) Representative confocal microscopy images of Iba1 and Aβ immunoreactivity in the hippocampus at ×20 magnification. (FIG. 26D) Numbers of Iba1⁺ cells around the surface of Aβ plaques were given on the right panel (n=6). (FIG. 26E) Representative blots of CD86, iNOS, and IL-1β protein expression levels in the hippocampus. (FIG. 26F) Quantification of CD86 protein expression levels in the hippocampus (n=3). (FIG. 26G) Quantification of iNOS protein expression levels in the hippocampus (n=3). (FIG. 26H) Quantification of IL-1β protein expression levels in the hippocampus (n=3). Western blotting data were normalized to 3-actin. Scale bar: 200 μm. Error bars denote s.d. *, **, ***, and **** denote p<0.05, p<0.01, p<0.001, and p<0.0001, respectively. The statistical difference among groups was assessed with the parametric one-way ANOVA with post-hoc Bonferroni test.

FIG. 27A-FIG. 27B. Intravenous administration of NSC- and iNSC-EVs inhibits astrocyte activation in 5×FAD mouse brains. (FIG. 27A) Representative confocal microscopy images of GFAP immunoreactivity in the prefrontal cortex at ×20 magnification. Numbers of immunoreactive cells in each group were given on the right panel (n=8). (FIG. 27B) Representative confocal microscopy images of GFAP immunoreactivity in the hippocampus at ×20 magnification. Numbers of immunoreactive cells in each group were given on the right panel (n=7). Scale bar: 200 μm. Error bars denote s.d. *, **, **** denote p<0.05, p<0.01, and p<0.0001, respectively. The statistical difference among groups was assessed with the parametric one-way ANOVA with post-hoc Bonferroni test.

FIG. 28A-FIG. 28F. Intravenous administration of NSC- and iNSC-EVs alters gene expression profiles in 5×FAD mouse hippocampal tissues. (FIG. 28A) Heatmap of all detected miRNAs in NSC-EVs and iNSC-EVs by microarray. (FIG. 28B) Volcano plot of all detected miRNAs in NSC-EVs and iNSC-EVs. (FIG. 28C) The top 10 miRNAs with highest readcounts in NSC-EVs. (FIG. 28D) The top 10 miRNAs with highest readcounts in iNSC-EVs. (FIG. 28E) The qRT-PCR validation of the top 10 miRNAs with highest readcounts in NSC-EVs. (FIG. 28F) The qRT-PCR validation of the top 10 miRNAs with highest readcounts in iNSC-EVs.

FIG. 29 shows that around 40% of RNA-seq-identified down-regulated genes in EV-injected groups, including pro-inflammatory genes (e.g., Il1b, Il12b) and Aβ production-related ones (e.g., Mmp13, Atp8b4), are predicted to be direct targets of EV-enriched miRNAs.

FIG. 30A-FIG. 30B. Intravenous administration of NSC- and iNSC-EVs alters gene expression profiles in 5×FAD mouse hippocampal tissues. Primary mouse microglia were stimulated by Aβ₁₋₄₂, followed by EV treatment and the transfection of either miR-9, let-7i, miR-21a, miR-34a, or miR-10b antagonists for 2 days. (FIG. 30A) The transcript levels of pro-inflammatory genes/1b and Nos2 in microglia were determined by qRT-PCR. (FIG. 30B) The transcript levels of anti-inflammatory genes CD206 and Ym1 in microglia were determined by qRT-PCR. Error bars denote s.d. n.s. denotes no significance. *, **, ***, and **** denote p<0.05, p<0.01, p<0.001, and p<0.0001, respectively. The statistical difference among groups was assessed with the parametric one-way ANOVA with post-hoc Bonferroni test.

FIG. 31A-FIG. 31B. Intravenous administration of NSC- and iNSC-EVs rescues neuronal loss in 5×FAD mouse brains. (FIG. 31A) Representative confocal microscopy images of NeuN immunoreactivity in the prefrontal cortex at ×20 magnification. Numbers of NeuN⁺ cells in each group were given on the right panel. (FIG. 31B) Representative confocal microscopy images of NeuN immunoreactivity in the hippocampus at ×20 magnification. Numbers of NeuN⁺ cells in each group were given on the right panel. N=4. Scale bar: 200 μm. Error bars denote s.d. * and ** denote p<0.05 and p<0.01, respectively. The statistical difference among groups was assessed with the parametric one-way ANOVA with post-hoc Bonferroni test.

FIG. 32A-FIG. 32D. Intravenous administration of NSC- and iNSC-EVs promotes neuroregeneration in 5×FAD mouse brains. (FIG. 32A-FIG. 32B) Representative confocal microscopy images of Ki67 (FIG. 32A) and DCX (FIG. 32B) immunoreactivity in the SVZ at ×20 magnification. Numbers of immunoreactive cells in each group were given on the right panel. (FIG. 32C-FIG. 32D) Representative confocal microscopy images of Ki67 (FIG. 32C) and DCX (FIG. 32D) immunoreactivity in the hippocampus at ×20 magnification. Numbers of immunoreactive cells in each group were given on the right panel. N=4. Scale bar: 200 μm. Error bars denote s.d. n.s. denotes no significance. *, **, **** denote p<0.05, p<0.01, and p<0.0001, respectively. The statistical difference among groups was assessed with the parametric one-way ANOVA with post-hoc Bonferroni test.

FIG. 33A-FIG. 33X. (FIG. 33A-FIG. 33D) Water maze: (FIG. 33A) Swim paths of mice while the platform was present (learning phase) or removed (memory phase); (FIG. 33B) Escape latencies in learning phase; (FIG. 33C) Platform crossing numbers and swimming distance in the target quadrant in memory phase (n=9). (FIG. 33D) Y-maze: novel arm entry numbers and ratios (n=9). (FIG. 33E) Open field test: rearing numbers, freezing times, and central field entry numbers (n=9). (FIG. 33F) Aβ immunoreactivity in the PFC. (FIG. 33G) Aβ plaque density and average size were quantified using ImageJ (n=8). (FIG. 33H) ELISA assay for the levels of Aβ₁₋₄₂ in the PFC (n=6). (FIG. 33I) pTau immunoreactivity in the PFC. (FIG. 33J) pTau signal intensity was quantified using ImageJ (n=7). (FIG. 33K) Representative images of Golgi-stained PFC tissues (up) and high-power magnification images showing the somata and primary dendrites of neurons (down). (FIG. 33L) The dendritic length was quantified using ImageJ (n=9~10). (FIG. 33M) The spine density was quantified using ImageJ (n=9~10). (FIG. 33N) Heatmap of top 40 DEGs among groups. (FIG. 33O-FIG. 33P) The top 5 GO terms (FIG. 33O) and KEGG pathways (FIG. 33P) of DEGs in comparison between NSC-EV- and PBS-injected mice. (FIG. 33Q-FIG. 33R) The top 5 GO terms (FIG. 33Q) and KEGG pathways (FIG. 33R) of DEGs in comparison between iNSC-EV- and PBS-injected mice. (FIG. 33S) Iba1 immunoreactivity in the PFC. (FIG. 33T) Numbers of Iba1⁺ cells were quantified using ImageJ (n=5). (FIG. 33U) Iba1 and Aβ immunoreactivities in the PFC. (FIG. 33V) Numbers of Iba1⁺ cells around the surface of Aβ plaques were quantified using ImageJ (n=6). (FIG. 33W-FIG. 33X) Representative blots (FIG. 33W) and quantification of CD86, IL-1β, and iNOS expression levels (FIG. 33X) in the PFC (n=3). Error bars denote s.d. ns denotes no significance. *, **, ***, **** denote p<0.05, p<0.01, p<0.001, p<0.0001, respectively. The statistical difference among groups was assessed with the parametric one-way ANOVA with post-hoc Bonferroni test.

FIGS. 34. iNSC-EVs mitigate Aβ oligomer-induced cell damage. Human neuronal cell line SH-SY5Y cells were stimulated by Aβ oligomer, followed by EV treatment. The cell viability was determined by CCK8 assay. Error bars denote s.d. * and **** denote p<0.05 and p<0.0001, respectively. The statistical difference among groups was assessed with the parametric one-way ANOVA with post-hoc Bonferroni test.

FIGS. 35A-35B. iNSC-EVs abrogate Aβ oligomer-induced synaptic factors expression inhibition. Human neuronal cell line SH-SY5Y cells were stimulated by Aβ oligomer, followed by EV treatment. The transcript levels of post-synaptic gene PSD95 and pre-synaptic gene Synaptophysin in SH-SY5Y cells were determined by qRT-PCR. Error bars denote s.d. n.s. denotes no significance. *, ***, and **** denote p<0.05, p<0.001, and p<0.0001, respectively. The statistical difference among groups was assessed with the parametric one-way ANOVA with post-hoc Bonferroni test.

FIGS. 36A-36B. iNSC-EVs reverse Aβ oligomer-induced oxidative stress and ferroptosis. Human neuronal cell line SH-SY5Y cells were stimulated by Aβ oligomer, followed by EV treatment. (A) ROS accumulation in SH-SY5Y cells was determined by quantification of ROS+ cells (DHE+ cells). (B) The transcript levels of anti-ferroptotic gene GPX4 and pro-ferroptotic gene ACSL4 in SH-SY5Y cells were determined by qRT-PCR. Error bars denote s.d. n.s. denotes no significance. *, **, ***, and **** denote p<0.05, p<0.01, p<0.001, and p<0.0001, respectively. The statistical difference among groups was assessed with the parametric one-way ANOVA with post-hoc Bonferroni test.

FIGS. 37A-37B. iNSC-EVs inhibit AP processing genes. Human neuronal cell line SH-SY5Y cells were stimulated by Aβ oligomer, followed by EV treatment. The transcript levels of key Aβ processing genes APP and BACE1 in SH-SY5Y cells were determined by qRT-PCR. Error bars denote s.d. n.s. denotes no significance. *** and **** denote p<0.001 and p<0.0001, respectively. The statistical difference among groups was assessed with the parametric one-way ANOVA with post-hoc Bonferroni test.

FIGS. 38A-38B. miR-26a-5p targets the 3′UTR of APP and ACSL4 transcripts. FIG. 38A The predicted consequential pairing of 3′UTR of candidate genes and miR-26a-5p on the TargetScan website. FIG. 38B Repression of luciferase activities by the APP and ACSL4 3′UTR were dependent on miR-26a-5p. Firefly luciferase activities were normalized to the internal control, Renilla luciferase activities. Error bars denote s.d. n.s. denotes no significance. ** and *** denote p<0.01 and p<0.001, respectively. The statistical difference among groups was assessed with the parametric one-way ANOVA with post-hoc Bonferroni test.

FIGS. 39A-39B. miR-26a-5p targets the 3′UTR of APP and ACSL4 transcripts. Human neuronal cell line SH-SY5Y cells were stimulated by Aβ oligomer, followed by the treatment of EVs with/without miR-26a-5p. The transcript levels of APP and ACSL4 in SH-SY5Y cells were determined by qRT-PCR. Error bars denote s.d. n.s. denotes no significance. **, ***, and **** denote p<0.01, p<0.001, and p<0.0001, respectively. The statistical difference among groups was assessed with the parametric one-way ANOVA with post-hoc Bonferroni test.

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

In a first aspect, the present invention provides a method for treating Alzheimer's disease in a patient in need thereof, comprising administration to the patient of a composition comprising a therapeutically effective amount of extracellular vesicles (EVs).

As used herein, a “therapeutically effective amount” refers to a sufficient amount of EVs for suppressing deleterious effects of Alzheimer's disease in a patient, at a reasonable benefit/risk ratio applicable to any medical treatment. It will be understood, however, that the total daily usage of EVs may be decided by the attending physician within the scope of sound medical judgment. The specific therapeutically effective dose level for any particular subject will depend upon a variety of factors including the disorder being treated and the severity of the disorder; the specific composition employed, the age, body weight, general health, sex and diet of the subject; the time of administration, route of administration, and rate of excretion of EVs employed; the duration of the treatment; drugs used in combination or coincidental with EVs; and like factors well known in the medical arts. For example, it is well known within the skill of the art to start doses of the composition at levels lower than those required to achieve the desired therapeutic effect and to gradually increase the dosage until the desired effect is achieved. In addition, a “therapeutically effective amount” is the amount that will elicit the biological or medical response of a tissue, system, or subject that is being sought by a researcher or clinician, and in particular elicit some desired therapeutic or prophylactic effect for suppressing deleterious effects of Alzheimer's disease in a patient.

One of skill in the art recognizes that an amount may be considered therapeutically “effective” even if the condition is not totally eradicated or prevented, but it or its symptoms and/or effects are improved or alleviated partially in the subject. Various indicators for determining the effectiveness of a method for suppressing deleterious effects of Alzheimer's disease in a patient are known to those skilled in the art.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

In some embodiments, the composition comprises from about 5% to about 10%, 20%, 30%, 40%, 50%, 60%, 70%, 80%, 90%, or 95% by weight of EVs, and preferably from about 30% to about 90% by weight of EVs, based upon the total weight of the composition taken as 100% by weight.

Other ingredients may be included in the claimed composition, such as other active agents, preservatives, buffering agents, salts, a pharmaceutically acceptable carrier, or other pharmaceutically acceptable ingredients.

As used herein, a “carrier” refers to a compound that facilitates the incorporation of a composition into cells or tissues. For example, without limitation, dimethyl sulfoxide (DMSO), Ethanol (EtOH), or PEG400 is a commonly utilized carrier that facilitates the uptake of many organic compositions into cells or tissues of a subject.

In some embodiments, the extracellular vesicles (EVs) are isolated from culture medium of cells. EVs are small bilipid layer-enclosed vesicles that display blood-brain barrier (BBB) penetrating ability and similar potency to their parental cells (Xia, X. et al., Transl Neurodegener. 11(1):53 (2022)).

In some embodiments, the cells are neural stem cells (NSCs), mesenchymal stem cells (MSCs), or fibroblast derived induced NSCs (iNSCs).

The inventors have obtained mouse fibroblast-derived induced NSCs (iNSCs) through somatic cell reprogramming, opening a new window for obtaining EVs derived from NSC-like cells (Ma, Y. et al., Cell Commun Signal. 17(1):96 (2019); Gao, G. et al. Induced neural stem/progenitor cell-derived extracellular vesicles promote recovery post-stroke. Clin Transl Med. 12(6):e936 (2022)). However, whether iNSC-derived (iNSC-EVs) can serve as an AD treatment remains unclear.

In some embodiments, the extracellular vesicles comprise one or more of following miRNAs: let-7, miR-9, miR-21, miR-34a, and miR-10b.

In some embodiments, the extracellular vesicles comprise let-7, miR-21, and miR-10b.

In some embodiments, the administration is at least once a day or more times a day.

In some embodiments, the administration is at least once in three days.

In some embodiments, the administration is at least for a month.

Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. One may also administer the composition in a local rather than systemic manner, for example, via injection of the composition directly into the infected area, often in a depot or sustained release formulation. Furthermore, one may administer the composition in a targeted drug delivery system, for example, in a liposome coated with a tissue specific antibody. The liposomes will be targeted to and taken up selectively by the organ.

In some embodiments, the administration is through various routes selected from oral, intravenous injectable, intramuscular injectable, intraperitoneal, intranasal, rectal, or sublingual route.

“Intraperitoneal” as used here means within or administered through the peritoneum. The peritoneum is a thin, transparent membrane that lines the walls of the abdominal (peritoneal) cavity and contains/encloses the abdominal organs such as the stomach and intestines.

As used herein, “sublingual” refers to situated or applied under the tongue.

In some embodiments, the administration of the composition is by a dose in the range of 2-2,000 mg, 2-500 mg, 2-200 mg, 2-150 mg, 5-100 mg, or 5-50 mg.

In some embodiments, the dose is administered in divided doses or a single dose.

In some embodiments, the administration of the composition is with a dose of EVs in the range of 2-2000 mg. In some embodiments, the administration of the composition is by oral with a daily dose of EVs in the range of 5-500 mg. In some embodiments, the administration of the composition is by oral with a daily dose of EVs in the range of 5-25 mg.

The dosage may range broadly, depending upon the desired effects and the therapeutic indication. The daily dosage regimen for an adult human patient may be, for example, an oral dose of between 0.01 mg and 3000 mg of EVs, preferably between 1 mg and 700 mg, e.g. 5 to 200 mg, or between about 0.1 mg and about 1,000 mg of EVs per kg of body weight of the subject. The dosage may be a single one or a series of two or more given in the course of one or more days, as is needed by the subject. In some embodiments, the compositions are administered for a period of continuous therapy, for example for a week or more, or for months or years. In some embodiments, EVs, or a pharmaceutically acceptable salt thereof, can be administered less frequently compared to the frequency of administration of an agent within the standard of care. In some embodiments, EVs, or a pharmaceutically acceptable salt thereof, can be administered one time per day. In some embodiments, the total time of the treatment regime with EVs, or a pharmaceutically acceptable salt thereof, can be less compared to the total time of the treatment regime with the standard of care.

In instances where human dosages for EVs have been established for at least some condition, those same dosages may be used, or dosages that are between about 0.1% and 500%, more preferably between about 25% and 250% of the established human dosage. Where no human dosage is established, as will be the case for newly discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

As used herein, the term “ED50” refers to the dose that produces the desired effect in 50% of the population, or median effective dose.

Dosage amount and interval may be adjusted individually to provide plasma levels of the active moiety that are sufficient to maintain the modulating effects, or minimal effective concentration (MEC). Dosages necessary to achieve the MEC will depend on individual characteristics and route of administration. However, HPLC assays or bioassays can be used to determine plasma concentrations. Dosage intervals can also be determined using MEC value. Compositions should be administered using a regimen that maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

In some embodiments, the composition down-regulates levels of Aβ1-42 in prefrontal cortex (PFC) and hippocampus.

In some embodiments, the composition reduces phosphorylated Tau (pTau) propagation.

In some embodiments, the composition regulates neuroinflammation.

In some embodiments, the composition reduces levels of CD86, iNOS, and IL-1β in the PFC and hippocampus. Specifically, CD86 refers to T-Lymphocyte Activation Antigen CD86; iNOS, Nitric Oxide Synthase 2; and IL-1β, Interleukin 1 Beta.

In some embodiments, the composition reduces one or more of following gene expression: Il1b, Il12b, Mmp13, and Atp8b4.

In a second aspect, the present invention provides a composition comprising therapeutically effective amount of extracellular vesicles for treating Alzheimer's disease in a patient in need thereof.

In a third aspect, the present invention provides a method for preparing any one of the compositions disclosed herein.

In a fourth aspect, the present invention provides use of any one of the compositions disclosed herein for treating Alzheimer's disease in a patient in need thereof.

In some embodiments, the composition is prepared in a form of pharmaceutical composition.

The term “pharmaceutical composition” refers to a mixture of EVs with other chemical components, such as diluents or carriers. The pharmaceutical composition facilitates administration of the composition to an organism. Pharmaceutical compositions can also be obtained by reacting compositions with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration. A pharmaceutical composition is suitable for human and/or veterinary applications.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compositions described herein are known to those skilled in the art.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes.

The pharmaceutical compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include EVs formulated in a compatible pharmaceutical carrier may also be prepared, placed in an appropriate container, and labeled for treatment of an indicated condition.

### DEFINITION

Unless defined otherwise, all technical and scientific terms used herein have the same meaning as is commonly understood by one of ordinary skill in the art. In the event that there are a plurality of definitions for a term herein, those in this section prevail unless stated otherwise.

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “individual,” “patient,” or “subject” are used interchangeably. None of the terms require or are limited to situation characterized by the supervision (e.g., constant or intermittent) of a health care worker (e.g. a doctor, a registered nurse, a nurse practitioner, a physician's assistant, an orderly, or a hospice worker).

Control: A control is an individual or a group of samples used as a standard of comparison for checking the results of a survey or experiment. In some context, a control is expressed as a reference.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The terms “treatment” and “treating,” as used herein, refer to an approach for obtaining beneficial or desired results including, but not limited to, a therapeutic benefit and/or a prophylactic benefit. For example, a treatment can comprise administering a system or cell population disclosed herein. A therapeutic benefit can refer to any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

A “therapeutic effect” may occur if there is a change in the condition being treated. The change may be positive or negative. A “change” in the condition being treated, may refer to at least 1%, 2%, 3%, 4%, 5%, 6%, 7%, 8%, 9%, 10%, 25%, 50%, 75%, or 100% change in the condition. The change can be based on improvements in the severity of the treated condition in an individual, or on a difference in the frequency of improved conditions in populations of individuals with and without the administration of a therapy. The term “therapeutically effective” should be understood to have a definition corresponding to ‘having a therapeutic effect.

Any titles or subheadings used herein are for organization purposes and should not be used to limit the scope of embodiments disclosed herein.

All publications and patent applications cited in this specification are herein incorporated by reference as if each individual publication or patent application were specifically and individually indicated to be incorporated by reference.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Materials and Methods

### Mice

5×FAD and C57BL/6 mice were purchased from Shanghai Model Organisms Center, Inc. All mice were housed in isolated ventilated cages (maximum five mice per cage) in the Comparative Medicine animal facilities of Tongji University School of Medicine (TUSM). All mice were maintained on a 12/12-hours light/dark cycle, 24~26 °C, 40~70% humidity with sterile pellet food and water ad libitum. Cages were checked daily to ensure animal welfare. All procedures were conducted according to protocols approved by the Institutional Animal Care and Use Committee (IACUC) of TUSM (reference number: SYXK (HU) 2014-0026).

### Isolation of Mouse NSCs

NSCs were isolated from C57BL/6 mouse fetal brains. Briefly, NSCs were obtained from embryonic day 13.5 mouse cortical tissues and cultured with NSC culture medium containing NeuroCult® NSC Basal Medium (Stem Cell Technologies), NeuroCult® NSC Proliferation Supplements (Stem Cell Technologies), 20 ng/mL FGF2 (BioWalkersville), 20 ng/mL EGF (BioWalkersville) and 2 μg/mL heparin (Sigma), N2 supplement (Gibco), 2 mM L-glutamine (ThermoFisher), 100 U/ml penicillin & streptomycin (ThermoFisher) to form neurospheres. Primary neurospheres were then dissociated into single cells and re-plated for next round of neurosphere formation. NSCs were purified after three rounds of neurosphere formation.

### Isolation of Mouse Microglia

Microglia were isolated from C57BL/6 mouse fetal brains. Briefly, postnatal day 1 mouse brains were dissected out and digested at 37 °C for 30 min in 0.25% trypsin-EDTA (Gibco) supplemented with 0.05% DNase I (Roche). Digestion was stopped by FBS (Invitrogen). Tissue sediment was centrifuged at 1500 rpm for 5 min at 4 °C. After trituration, dissociated cells were cultured in DMEM (Gibco) supplemented with 10 ng/mL GM-CSF and 10% FBS, 50 U penicillin and 50 mg/mL streptomycin at 37 °C. Culture dishes were coated with 100 mg/mL Poly-D-Lysine (Sigma) and 5 mg/mL Fibronectin (Sigma). The culture medium was replaced every 3 days. Mouse primary microglia in the glial cell-mixed cultures were induced to detach by shaking. Floating microglia were collected by 1500 rpm centrifugation for 5 min at 4 °C.

Reprogramming of Mouse Fibroblast into iNSCs

Mouse fibroblasts were derived from mouse embryos at embryonic day 14. Briefly, all the internal organs, head and spinal cord were removed from embryos. The remaining skin tissues were washed twice with PBS, and dissociated with 0.25% trypsin-EDTA solution. Mouse fibroblasts were cultured in high glucose medium supplemented with 10% FBS, 1% non-essential amino acid (non-AA), 100 U/ml penicillin, 100 μg/ml streptomycin at 37 °C in a 5% CO2 humidified atmosphere.

Mouse fibroblasts were directly reprogramed into iNSCs. Briefly, mouse fibroblasts were incubated in the mixed virus-containing supernatants overnight. 10 μg/mL polybrene (Millipore) was added to facilitate virus transfection. Infected fibroblasts were cultured in NSC culture medium 1 day after the second infection. NSC culture medium was replaced every two days. Day 28 after retroviral transduction, colonies were formed, manually picked and suspended into single cells to generate neurospheres. Floating primary neurospheres were collected after culturing for 4-6 days and re-plated into Poly-D-Lysine/Fibronectin-coated 6-well plates. Cells were collected after reaching 80% confluency and re-suspended into single cells for a second round of neurosphere formation. After 3 rounds of selection and enrichment, cells were collected for iNSC characterization.

### Aβ Oligomer Preparation and Aβ-Induced Microglial Activation In Vitro

Aβ₄₂ peptide solution (1 mM) was prepared by dissolving Aβ₄₂ peptide (GL Biochem, Shanghai) in 221.7 μl HFIP (1,1,1,3,3,3-hexafluoro-2-propanol) (Sigma-Aldrich). The solution was placed at room temperature (RT) for 1 h, and then kept on ice for 10 min. Afterwards, the solution was aliquoted into non-siliconized microcentrifuge tubes (100 μl solution containing 0.45 mg Aβ₄₂), and dried overnight at RT. The residues were dissolved in 20 μl dimethyl sulfoxide (DMSO), added with F12 medium to make a 100 μM stock solution, incubated at 4 °C overnight, and then centrifuged at 14,000×g for 10 min at 4 °C. The Aβ oligomers were presented in the supernatant, that was confirmed by immunoblot using anti-Aβ antibody (1:1000, BioLegend).

To mimic Aβ-induced microglial activation in vivo, primary microglia were treated with Aβ oligomer (dose: 10 μM) for 48 h at 37 °C. The activation of microglia was confirmed by examining the expression levels of transcripts corresponding to pro-inflammatory genes (Tnf and Il1b) and anti-inflammatory genes (CD206 and Ym1) in microglia, and that of pro-inflammatory cytokines (TNF-α and IL-1β) in the conditioned medium.

### Agonist/Antagonist/siRNA and Transfection

The antagomiR control, antagomiR-9, antagomiR-21a, antagomiR-34a, antagomiR-10b, and antagomir-let7i were purchased from GenePharma (GenePharma). Transfection of 20 nM antagomiR-9, antagomiR-21a, antagomiR-34a, antagomiR-10b, antagomir-let7i, or their corresponding controls was performed using the Lipofectamine 2000 reagent (Invitrogen) according to the manufacturer's instruction.

### Isolation of EVs

EVs were isolated from conditioned medium of NSCs and iNSCs culture. Briefly, conditioned media was first centrifuged at 300 g for 10 min to remove cells, at 3,000 g for 20 min to remove cellular debris, at 10,000 g for 30 min to remove intracellular organelles. The supernatant was filtered using a 0.22 μm syringe filter, and then centrifuged at 100,000 g for 4 h to collect EVs. Centrifugation was carried out at 4 °C. EVs were resuspended in PBS and stored in −80 °C for future use.

Staining of EVs with Dil

EV solution was gently mixed with Dil in PBS (1:1000) and incubated for 30 min in the dark at RT. The mixture was washed with PBS once, filtered with 0.22 μm filter, and ultracentrifuged at 100,000 g for 2 h at 4 °C, leaving any unbound dye to be discarded. Mice were given intravenous administration of Dil-labeled EVs or PBS. After 5 min, mice were sacrificed for brain tissues. Brain tissues were sectioned to brain slices. After sectioning 10 μM sections, slices were imaged on Zeiss AX10 fluorescence microscope to detect Dil signals in brain cells.

### Group Setting and Intravenous Administration of EVs to 5×FAD

Four-month-old 5×FAD mice were randomly divided into 3 groups: 5×FAD+PBS (n=10/group), 5×FAD+NSC-EVs (n=11/group), and 5×FAD+iNSC-EVs (n=11/group). Same age C57BL/6 mice injected with PBS (WT+PBS) were used as controls (n=10/group). 200 μl EVs (0.5 μg/μl concentration) or an equal volume of PBS control were administrated intravenously over 5 min via tail vein every three days. Each animal received 10 injections. The dosage of EVs was chosen based on the recent stroke studies (Leng, F., Edison, P. Neuroinflammation and microglial activation in Alzheimer disease: where do we go from here? Nat Rev Neurol. 17(3):157-172 (2021)).

### Nanoparticle Tracking Analysis (NTA)

NTA analyses were carried out to determine the sizes and concentrations of EV samples. Briefly, EVs were resuspended in 1 ml PBS for NTA analyses at defined conditions of the measurements (25 °C, 1 cP viscosity, 25 sec per capture frame, and 60 sec measurement time). NTA analyses were assessed on NanoSight NS300 system (Malvern Instruments, UK) with a sCMOS camera. Three individual measurements were applied for measuring the sizes and concentration of EVs.

### Electron Microscopy (EM)

For scanning electron microscopy (SEM), NSCs and iNSCs cultured on glass coverslips were fixed with 2.5% glutaraldehyde and washed three times with PBS. Cells were dehydrated in a series of increasing ethanol concentrations and transferred for critical drying. Cells were then coated with gold-palladium to increase the image contrast and imaged using scanning electron microscope (S-3400, Hitachi). For transmission electron microscopy (TEM), purified EVs were negatively stained and then spread on the copper grids. The droplets of EVs were removed with filter paper and air-dried at RT. Images were taken using transmission electron microscopy (JEM-1230, JEOL Ltd.).

### Morris Water Maze (MWM) Test

MWM was performed to determine the cognitive function and memory of mice. Mice were introduced into a circular, water-filled tank which was equally divided into four quadrants. Visual cues were placed around the pool in plain sight of the mouse to flag the submerged platform. Various parameters of mouse movement were recorded, including the time spent in each quadrant of the pool, the time taken to reach the platform (escape latency), and the total distance travelled. For each trial, the mouse was allowed no more than 60 sec to find the submerged platform before they were guided to the platform, removed from water, towel dried, and returned to their cage. Each mouse completed 4 trials per day during the 6-day training phase. One day after the training, the probe test was conducted. The platform was removed and each mouse was given 60 sec to swim in the water. The swimming was videotaped and analyzed by Ethovision XT (Noldus, Netherlands).

### Y Maze

The Y-maze had three arms (20 cm long×10 cm wide×20 cm high) at 120° angles. The three arms included the start arm (always open); the novel arm, which is blocked at the first trial, but opened at the second trial; and the other arm (always open). In the first trial, one of the arms is blocked and the animal is allowed to explore the rest two arms for 10 min. The second trial was conducted 2 h after the first trial. In the second trial, the blocked arm is opened and considered as the novel arm. The animal is allowed to explore all three arms for 5 min and the entrance to each arm is recorded. The spatial memory of animals can be revealed by a discrimination to the novel arm in the second trial. A video camera linked to the Any-Maze animal tracking system software was mounted above the maze, and used to record the movements of the mice for analysis. The time spent in and entries into the novel arms indicated the spatial recognition memory (learned behavior). The experimental environment was kept quiet, light, slightly dark and each of the arms of the Y maze was cleaned with 70% ethanol solution between trials.

### Open Field Test

Open field test was performed using an open field apparatus (30×30×21 cm) with nine virtual quadrants (10×10 cm each). Central area is a region consisting of four sub squares marked with red color. Each mouse was put in the center at start of test and permitted to freely explore for 5 min. Number of crossings, time spent in the center and periphery, rearing, fecal pallets, time of immobility, jumping, and efforts made by each mouse to getaway were recorded. The total distance and time as well as the distance travelled and time spent in the center zone (10 cm×10 cm) were analyzed using the Activity Monitor software (Med Associates, Inc.). The apparatus was cleaned with 70% ethanol between trials.

### Fear Conditioning Test

The test consists of a training phase and test phase, which took place on two consecutive days. During the training phase, mice were placed in the conditioning chamber for a 120 sec accommodation period. The mice were then presented with an 80 dB tone for 30 s, co-terminating with a two second 0.7 mA foot shock, followed by another 120 s interval and a second tone and foot-shock pairing. Mice were removed from the chamber thirty seconds after the second shock. In the context test, mice were placed in the same chamber used during training for 5 min without a tone or footshock, and the duration of freezing behavior was recorded using a stopwatch. The percentage freezing time (defined as the time in which mice had no movements except for respiration) was recorded and analyzed by the Panlab Startle and Fear combined system with Packwin 2.0 software.

### Protein Extraction and Western Blotting

Brains tissues were removed from euthanized mice and homogenized by a homogenizer in the M-PER Protein Extraction Buffer (Pierce) containing a protease inhibitor cocktail (Sigma). Protein concentrations were determined using BCA Protein Assay Kit (Pierce). Proteins (5~10 μg) from tissue lysates were separated by sodium dodecyl sulfate-polyacrylamide gel electrophoresis (SDS-PAGE) and electrophoretic transferred to polyvinyldifluoridene membranes (Millipore and Bio-Rad). Proteins were treated with purified primary antibodies for CD9 (rabbit, Abcam, 1:2000), Flotillin1 (mouse, BD Biosciences; 1:5000), Flotillin2 (rabbit, CST, 1:1000), APOA1 (rabbit, Affinity Biosciences; 1:500), APOA2 (rabbit, Affinity Biosciences; 1:500), CD68 (rabbit, Abcam; 1:1000), CD86 (rabbit, CST; 1:1000), or 3-actin (mouse, CST; 1:1000) overnight at 4 °C followed by a horseradish peroxidase-linked secondary anti-rabbit or anti-mouse antibody (Cell Signaling Technologies, 1:10,000). Antigen-antibody complexes were visualized by Pierce ECL Western Blotting Substrate (Thermo Fisher Scientific, Waltham, MA). Films were then scanned with a CanonScan 9950F scanner and the acquired images were analyzed on a Macintosh computer using the free public domain NIH ImageJ program.

### Enzyme-Linked Immunosorbent Assay (ELISA)

Mouse hippocampal and cortical tissue lysates were collected and commercially available ELISA kits were used to measure the levels of Aβ₁₋₄₂ (cat #KE1266, Immunoway), TNF-α (cat #KE1419, Immunoway), and IL-1β (cat #KE1416, Immunoway) according to manufacturer's protocols. Diluted standards and lysates in triplicate were added to the corresponding wells and incubated at RT for 2 h on a microplate shaker. Sample Diluent was used as blank control. Streptavidin-HRP was added (100 μl/well) and incubated at RT for 45 min. TMB Substrate Solution was then added (100 μl/well) and incubated at RT for 30 min. The enzyme reaction was stopped by Stop Solution (100 μl/well). Absorbance of each well was read using spectrophotometer DV8200 (Drawell). Aβ₁₋₄₂, TNF-α, and IL-1β concentrations were determined according to the standard curve.

### Immunohistochemistry

Tissue sections were fixed in 4% paraformaldehyde (Sigma) overnight at 4 °C and transferred into 30% sucrose to incubate at 4 °C for 24 h. Post 3 times of PBS washes, sections were incubated with permeabilizing and blocking buffer containing 5% goat serum (Vector Laboratories) and 0.5% Triton X-100 (Bio-Rad) in PBS at RT for 1 h. Tissue sections were then incubated with primary antibody for Aβ (mouse, cat #15126S, CST, 1:400), Nestin (chicken, cat #NB100-1604, Novus, 1:5000), Sox2 (Rabbit, cat #ab92494, Abcam, 1:500), CD9 (rabbit, cat #98327, CST, 1:100), Ki67 (rabbit, cat #9129S, CST, 1:400), DCX (rabbit, cat #4604S, CST, 1:400), NeuN (mouse, cat #MAB377, Millipore, 1:200), or Iba1 (goat, cat #ab5076, Abcam, 1:500) overnight at 4 °C. The next day, tissue sections were incubated with secondary antibodies (Molecular Probes) at RT for 1 h after washing with PBS for 3 times. Sections were mounted using Vecta-Shield (Vector Laboratories). Images were taken using a Zeiss AX10 fluorescence microscope accompanied with ZEN 2.3 (blue edition) software. For quantification of immunoreactivity positive cell density, the hippocampus, prefrontal cortex (PFC), and sub-ventricular zone (SVZ) were imaged at ×20 magnification. Three coronal sections spanning the hippocampus, PFC, and SVZ at different depths on the rostro-caudal axis were analyzed for each animal. Four to five images were captured on matching areas of selected brain regions per section. The numbers of immunoreactivity positive amyloid plaques and cells were quantified by ImageJ. The amyloid plaque burden (area occupied by all plaques divided by the total area) was estimated in the cortex and hippocampus for each section using the Analyze Particles plugin of ImageJ software. Values from each section were averaged to acquire a mean immunoreactivity positive amyloid plaque and cell density for each animal.

### Golgi-Cox Staining

Mouse cortical and hippocampal tissues were incubated at RT in Golgi-Cox staining solution A/B of FD Rapid Golgi Stain™ Kit (FD Neurotechnologies) for 14 d protected from light. The solution C was replaced the following day and brain tissues were incubated in solution C at 4 °C for 72 h in the dark. Afterwards, brain tissue blocks were included in a 10% sucrose-4% agarose solution, obtaining 100 μm thick sections in a Leica VT1200S vibratome. Sections were mounted on gelatin-coated glass slides and naturally dried for 20 min. The Golgi-Cox reaction was performed following the manufacturer's instructions. Sections were finally counterstained with toluidine blue to identify anatomical structures. Images were taken at 20, 40, and 64 objectives using Olympus1X71.

Quantitative Reverse Transcription Polymerase Chain Reaction (qRT-PCR)

The mRNAs and miRNAs were isolated from brain tissues and cells using RNeasy mini kit (Qiagen) according to the manufacturer's instructions. Genomic DNA was removed using DNase I digestion kit (Qiagen). cDNA was synthesized using miScript II RT kit (Qiagen). Transcripts were amplified using SYBR green PCR kit (Qiagen) with the AB17500 (Applied Biosystems) with specific primer sets for Gapdh (Forward: CATGTTCCAGTATGACTCCACTC (SEQ ID NO: 1), Reverse: GGCCTCACCCCATTTGATGT (SEQ ID NO: 2), Il1b (Forward: CCAGCAGGTTATCATCATCATCC (SEQ ID NO: 3), Reverse: CTCGCAGCAGCACATCAAC (SEQ ID NO: 4)), Nos2 (Forward: CCCTTCAATGGTTGGTACATGG (SEQ ID NO: 5), Reverse: ACATTGATCTCCGTGACAGCC (SEQ ID NO: 6)), Tnf (Forward: ACGTGGAACTGGCAGAAGAG (SEQ ID NO: 7), Reverse: GGTCTGGGCCATAGAACTGA (SEQ ID NO: 8)), Myod88 (Forward: CGGGTCCCTGGACTCCTTCA (SEQ ID NO: 9), Reverse: CGCGTTTCCAGCTCTCGGAT (SEQ ID NO: 10)), Ym1 (Forward: TCACAGGTCTGGCAATTCTTCTG (SEQ ID NO: 11), Reverse: ACTCCCTTCTATTGGCCTGTCC (SEQ ID NO: 12)), CD206 (Forward: TCTTTGCCTTTCCCAGTCTCC (SEQ ID NO: 13), Reverse: TGACACCCAGCGGAATTTC (SEQ ID NO: 14)), and miRNAs (Universal primer: GAATCGAGCACCAGTTACGC (SEQ ID NO: 15), U6 primer: TGGCCCCTGCGCAAGGATG (SEQ ID NO: 16), miR-9: TCTTTGGTTATCTAGCTGTATGA (SEQ ID NO: 17), let-7i: TGAGGTAGTAGTTTGTGCTGTT (SEQ ID NO: 18), miR-10b: TACCCTGTAGAACCGAATTTGTG (SEQ ID NO: 19), miR-21a: TAGCTTATCAGACTGATGTTGA (SEQ ID NO: 20), miR-30a: TGTAAACATCCTCGACTGGAAG (SEQ ID NO: 21), miR-99a: AACCCGTAGATCCGATCTTGTG (SEQ ID NO: 22), let-7f: TGAGGTAGTAGATTGTATAGTT (SEQ ID NO: 23), let-7g: TGAGGTAGTAGTTTGTACAGTT (SEQ ID NO: 24), miR-181a: AACATTCAACGCTGTCGGTGAGT (SEQ ID NO: 25), miR-26a: TTCAAGTAATCCAGGATAGGCT (SEQ ID NO: 26), miR-129: CTTTTTGCGGTCTGGGCTTGC (SEQ ID NO: 27), let-7c: TGAGGTAGTAGGTTGTATGGTT (SEQ ID NO: 28), let-7b: TGAGGTAGTAGGTTGTGTGGTT (SEQ ID NO: 29), miR-148a: TCAGTGCACTACAGAACTTTGT (SEQ ID NO: 30), miR-100: AACCCGTAGATCCGAACTTGTG (SEQ ID NO: 31)). Reactions were run in triplicates for each sample and no-template blanks were used as negative controls. Values were normalized to the Gapdh (for mRNA) and U6 snRNA (for miRNA).

### RNA Sequencing (RNA-Seq)

Total RNA was extracted from cortical and hippocampal tissues of experiment and control mice using the mirVana miRNA Isolation Kit (Ambion). Sample processing was carried out by OE Biotech Co., Ltd (Shanghai, China). RNA integrity was evaluated using the Agilent 2100 Bioanalyzer (Agilent Technologies). The libraries were constructed using TruSeq Stranded mRNA LTSample Prep Kit (Illumina). Libraries were sequenced on the Illumina sequencing platform (Illumina NovaSeq 6000) and 125 bp/150 bp paired-end reads were generated. Raw data (raw reads) were processed using Trimmomatic. Clean reads were mapped to reference genome using hisat 2. FPKM value of each gene was calculated using cufflinks and the read counts of each gene were obtained by htseq-count DEGs were identified using the DESeq 2012 R package functions estimateSizeFactors and nbinomTest. P value <0.05 and foldChange>2 or foldChange<0.5 was set as the threshold for significantly differential expression Hierarchical cluster analysis of differentially expressed genes (DEGs) was performed to explore genes expression pattern. The enrichment of Gene Ontology (GO) term and Kyoto Encyclopedia of Genes and Genomes (KEGG) pathways in DEGs were analyzed using DAVID online tools (david.abcc.ncifcrf.gov). GO terms corresponding to biological process were selected. The enrichment of GO terms and KEGG pathways was determined by the p-value from DAVID online tool analysis. The p-value were determined by a modified Fisher's exact test, adjusted by the Benjamini-Hochberg method.

miRNA Microarray

Total RNA was extracted from NSC-EVs and iNSC-EVs. Sequencing libraries were generated from 3 μg total RNA per sample as input material using NEBNext® Multiplex Small RNA Library Prep Set for Illumina® (NEB). The clustering of the index-coded samples was performed on a cBot Cluster Generation System using TruSeq SR Cluster Kit v3-cBot-HS (Illumia). After cluster generation, the library was sequenced on an Illumina Hiseq 2500/2000 platform, and 50 bp single-end reads were generated. Raw data (raw reads) of fastq format were firstly processed through custom perl and python scripts for quality control. The small RNA tags were mapped to reference sequence by Bowtie without mismatch to analyze their expression and distribution on the reference. Mapped small RNA tags were used to match with known miRNA. miRBase 20.0 was used as reference for known miRNA, miRDeep2 and sRNA-tools-cli were used to obtain novel miRNAs and draw the secondary structures, respectively. miRNA expression levels were estimated by TPM (transcript per million).

### Statistical Analyses

The statistical difference between two independent groups was analyzed with the unpaired Student's t-test, and that among more than two groups was assessed with the parametric one-way ANOVA with post-hoc Bonferroni test. Data were shown as mean±s.d., and significance was determined as p<0.05.

### Results

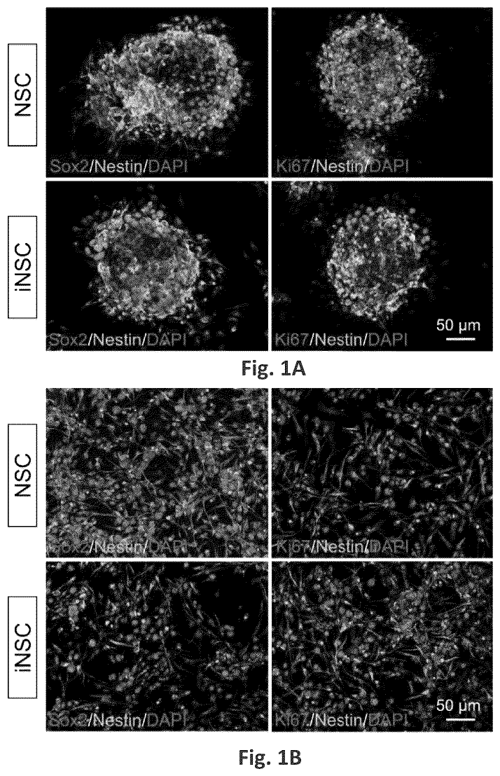

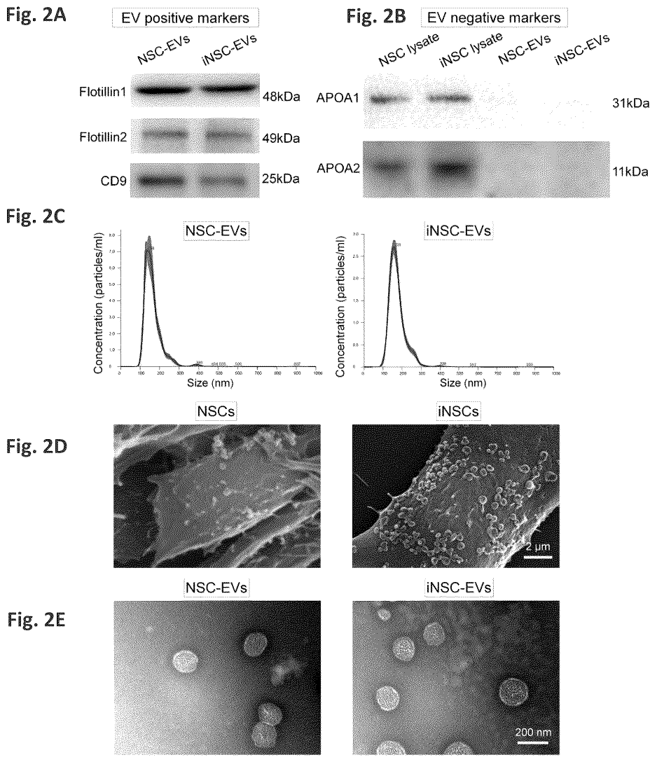

EVs were isolated from the conditioned medium of mouse NSCs and iNSCs. iNSCs were characterized by immunocytochemistry (FIG. 1A and FIG. 1B). The purity of EVs was validated by western blotting, nanoparticle tracking analysis, and electronic microscopy analyses (FIG. 2A-FIG. 2E).

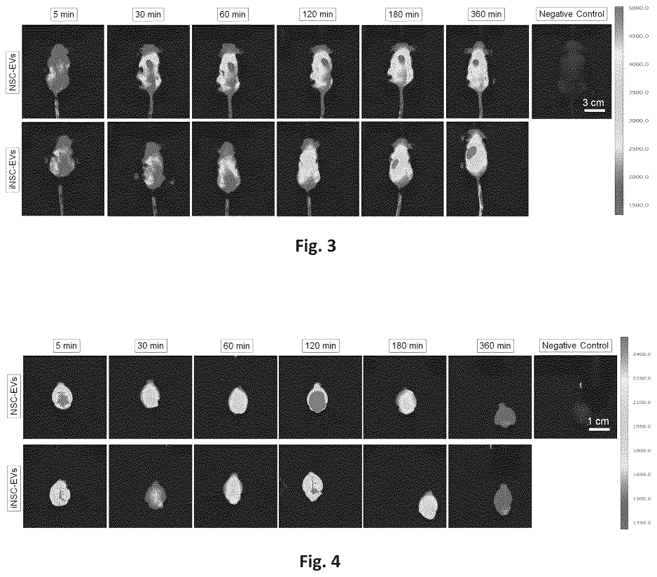

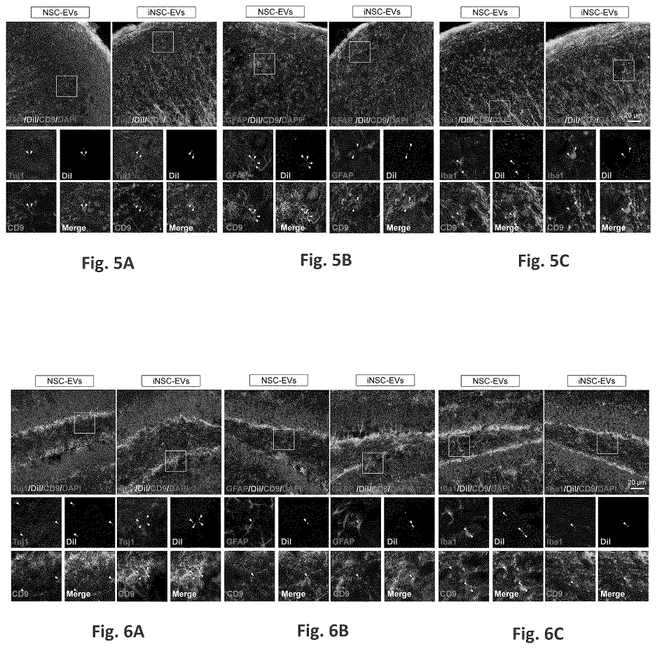

Five minutes after the intravenous administration of EVs labeled with a lipophilic carbocyanine fluorescent membrane labeling dye Dil, Dil signals were detected throughout mouse body (FIG. 3) and in the brains (FIG. 4) by in vivo fluorescence analyses. Immunohistochemical analyses detected Dil signals, colocalized with EV marker CD9, in Tuj1⁺ neurons, GFAP⁺ astrocytes, and Iba1⁺ microglia, in the cortex (FIG. 5A-FIG. 5C) and hippocampus (FIG. 6A-FIG. 6C), suggesting that EVs have been taken upby brain cells.

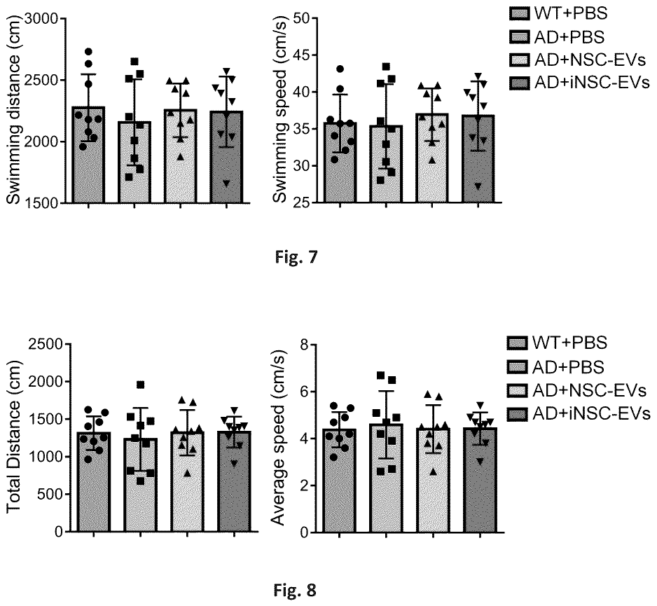

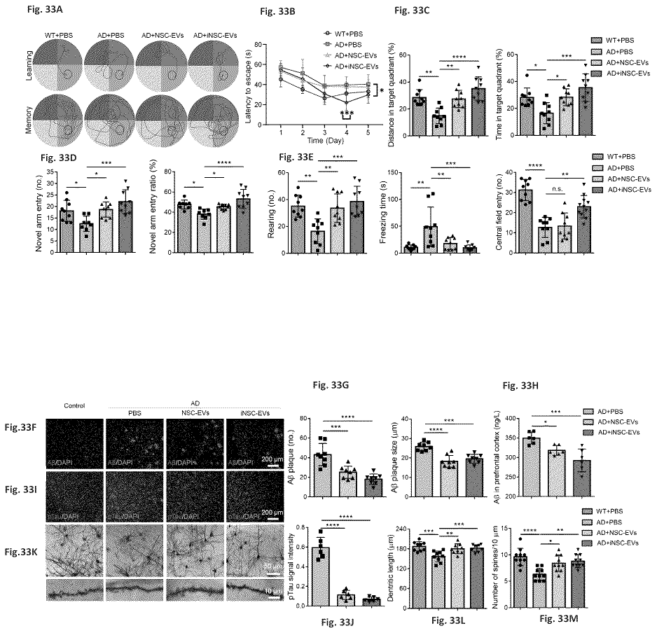

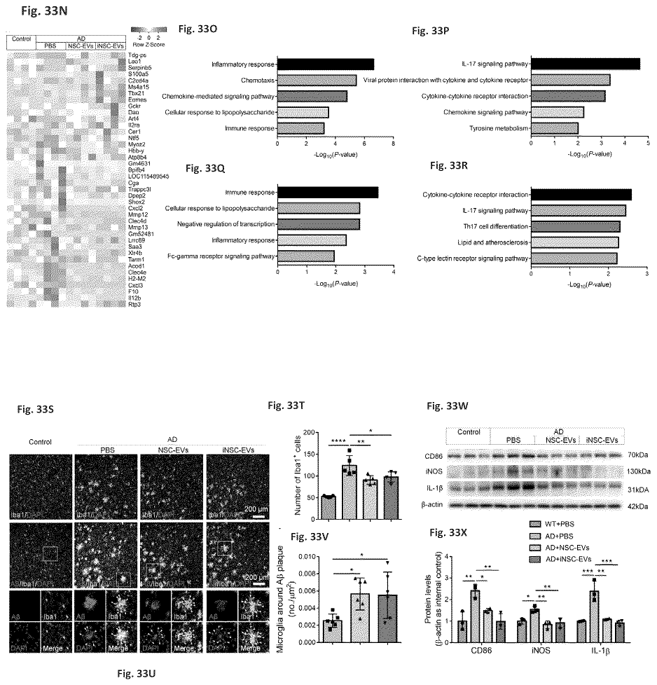

Four-month-old APP/PS1 double transgenic mice that co-express five familial AD mutations (5×FAD mice) were then intravenously administrated with either 200 μl EVs (0.5 μg/μl concentration) or an equal volume of PBS every three days for one month. After EVs treatment, 5×FAD mice spent shorter times to find the platform in water maze versus PBS controls (FIG. 33A-FIG. 33B). EV treatment also increased distance traveled and time spent of 5×FAD mice in the target quadrant in memory phase versus PBS controls (FIG. 33C), without affecting motor skills (FIG. 7).

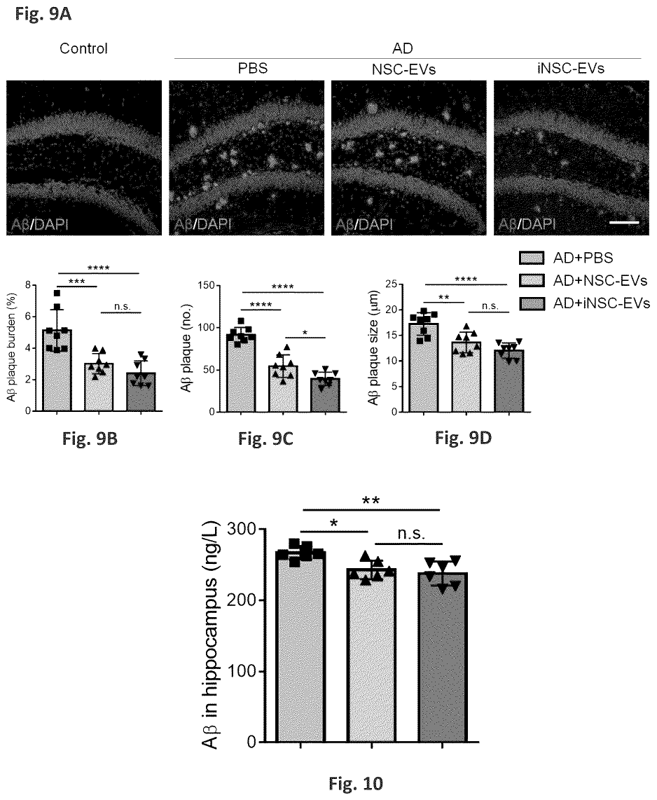

Y maze test showed that EVs treatment significantly increased the numbers and ratios of novel arm entries of 5×FAD mice versus PBS controls (FIG. 33D), without influencing motor performance of mice (FIG. 8). EVs treatment also significantly increased the rearing numbers, decreased freezing time, and expanded central area entry numbers of mice in open field test versus PBS controls (FIG. 33E). Moreover, EVs treatment significantly reduced extracellular plaque density and average size in the prefrontal cortex (PFC) (FIG. 33F, FIG. 33G) and hippocampus (FIG. 9A-FIG. 9D) of 5×FAD mice.

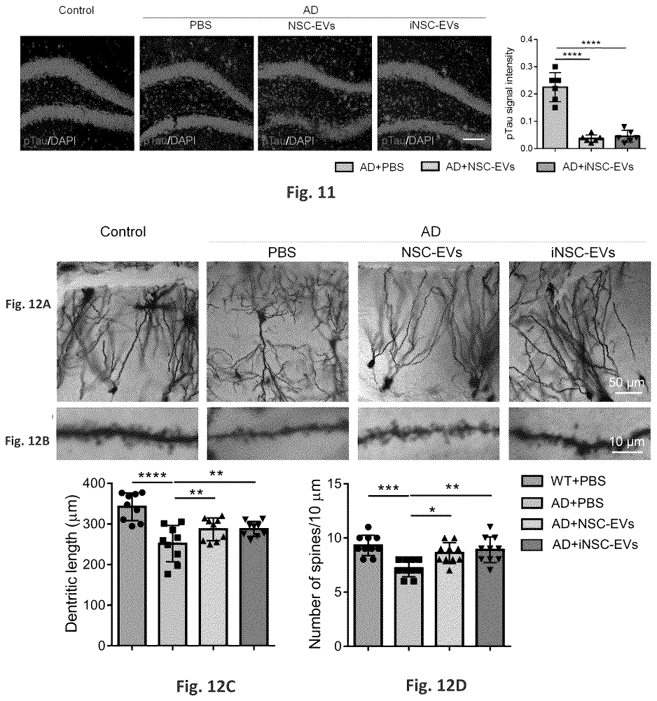

Importantly, ELISA assay showed significantly decreased levels of Aβ₁₋₄₂ in the PFC (FIG. 33H) and hippocampus (FIG. 10) of EV-injected mice. Similarly, EVs treatment reduced phosphorylated Tau (pTau) propagation (FIG. 33I, FIG. 33J, FIG. 11) and increased dendritic length and dendritic spine density (FIG. 33K, FIG. 33L, FIG. 33M, FIG. 12A-FIG. 12D) in the PFC and hippocampus of 5×FAD mice, compared with PBS group.

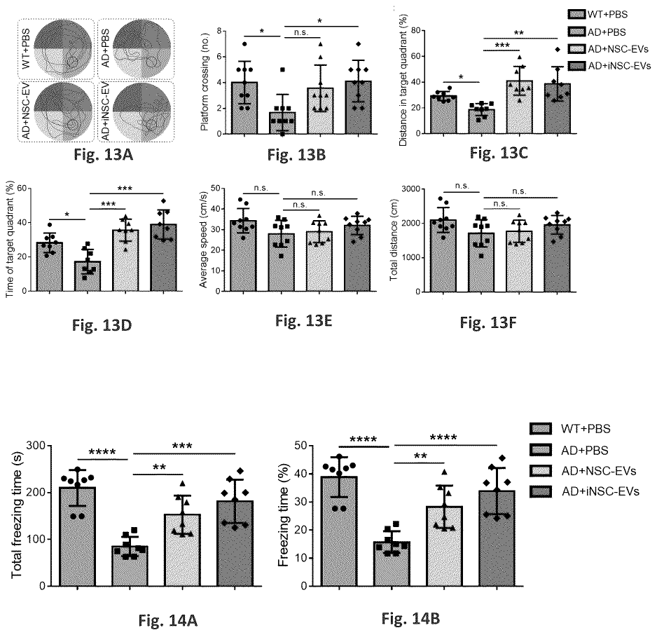

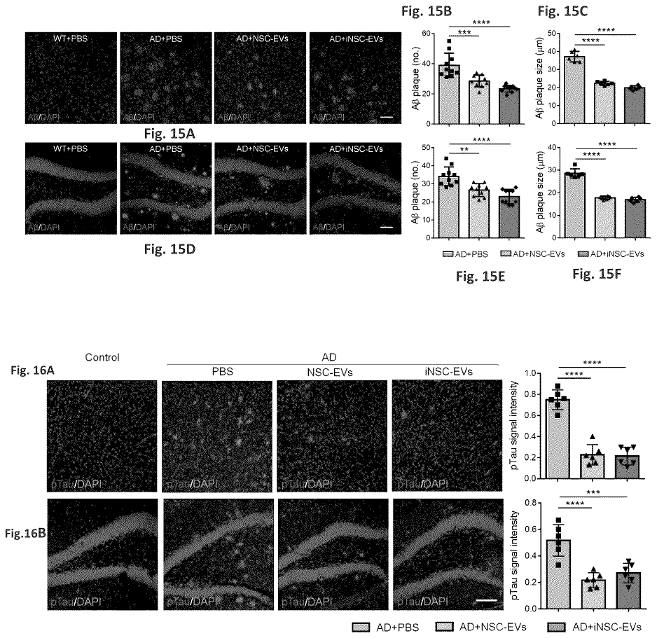

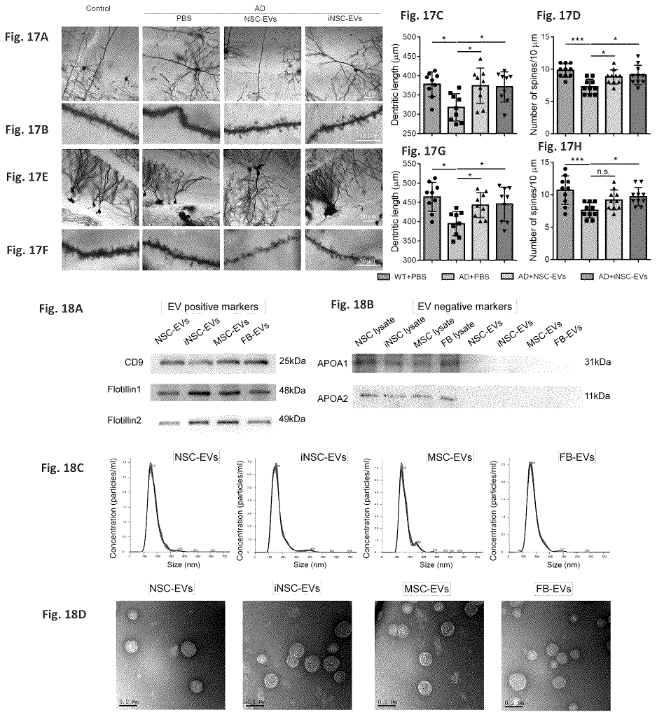

Moreover, EVs significantly improved the cognitive function of 5×FAD mice even 1 month post administration as shown in water maze and fear conditioning tests (FIG. 13A-FIG. 13F, FIG. 14A-FIG. 14B). Furthermore, Aβ₁₋₄₂ accumulation (FIG. 15A-FIG. 15F) and pTau propagation (FIG. 16A-FIG. 16B) were dramatically reduced, and dendritic length and dendritic spine density (FIG. 17A-FIG. 17H) were significantly increased in the PFC and hippocampus of EV-injected mice, compared with PBS controls 1 month post EV administration, suggesting that the therapeutic effects of EVs has a great duration. Hence, iNSC-EVs showed comparable (no less if not more) therapeutic potentials than NSC-EVs on AD-like behavioral and pathological phenotypes.

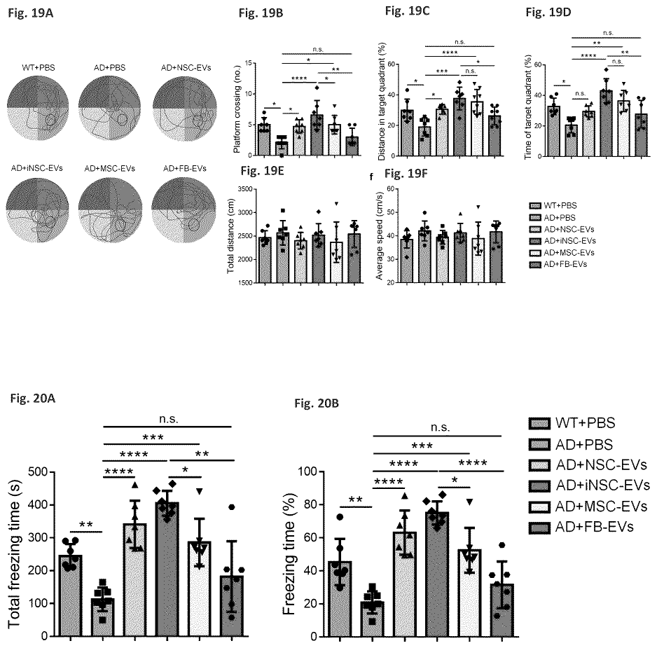

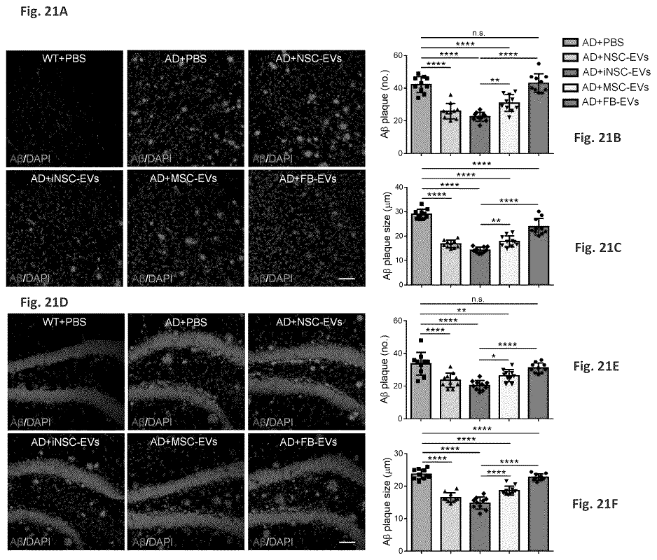

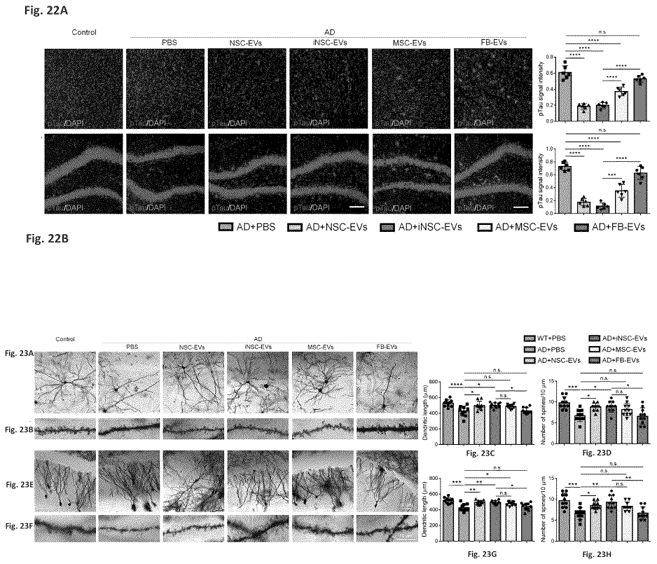

EVs derived from MSCs, a widely used cell type in regenerative medicine, and fibroblasts, the origin cell of iNSCs were further collected (FIG. 18A-FIG. 18D). Water maze test showed greater platform crossing numbers and similar distance/time in target platform in iNSC-EV-injected mice, compared with MSC derived-EV (MSC-EVs)- and fibroblast-derived EV (FB-EV)-injected groups (FIG. 19A-FIG. 19F). Fear conditioning test suggested greater effects of iNSC-EVs on increasing freezing times of 5×FAD mice, compared with MSC-EVs and FB-EVs (FIG. 20A-FIG. 20B). iNSC-EVs also exhibited greater inhibitory effects on Aβ₁₋₄₂ accumulation (FIG. 21A-FIG. 21F) and pTau propagation (FIG. 22A-FIG. 22B), and better therapeutic effects on restoring the dendritic length and dendritic spine density (FIG. 23A-FIG. 23H) in the PFC and hippocampus of 5×FAD mice versus MSC-EVs and FB-EVs. Therefore, iNSC-EVs showed greater potentials than MSC-EVs on alleviating AD-like phenotypes of 5×FAD mice.

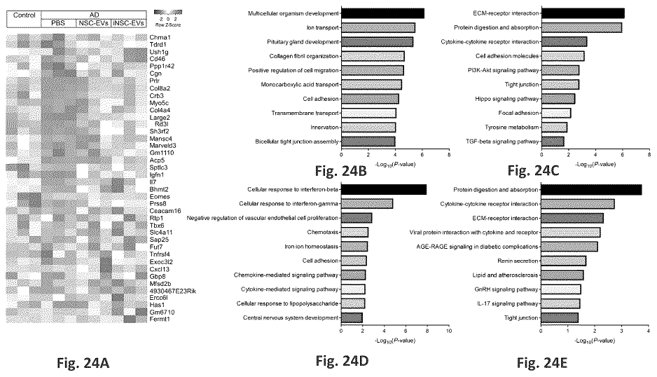

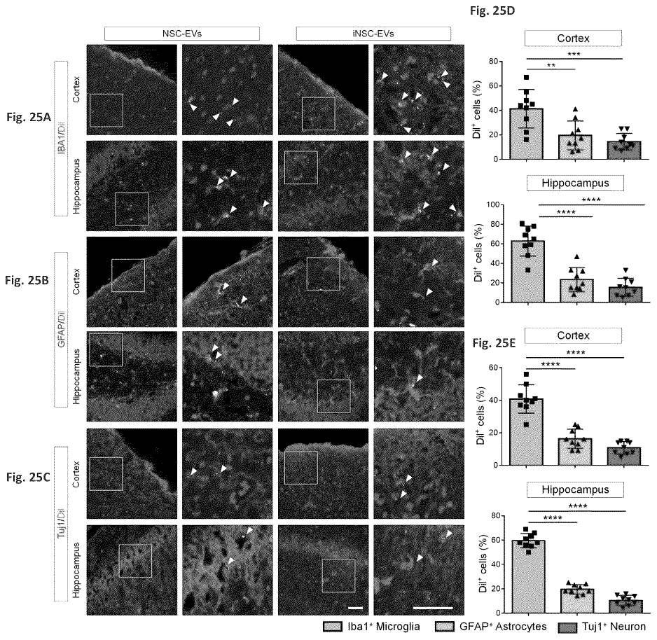

To understand the underlying mechanisms of iNSC-EV-mediated amelioration of AD-like phenotypes, RNA-seq analyses were carried out for RNAs isolated from PFC. The TOP 40 differentially expressed genes (DEGs) among groups revealed that AD-induced gene expression deregulation was restored by EVs administration (FIG. 33N). GO and KEGG analyses indicated a strong association of the DEGs in either NSC-EV- or iNSC-EV-injected groups versus PBS controls with inflammation-related terms and signaling pathways, respectively (FIG. 33O-FIG. 33R). Similar results were obtained via RNA-seq analyses for RNAs isolated from hippocampus, suggesting neuroinflammation, a key pathological feature and risk factors of AD, as a potential target of EVs treatment (FIG. 24A-FIG. 24E). RNA-seq results were corroborated by immunohistochemical analyses, showing that NSC-EVs and iNSC-EVs were mainly internalized by microglia, the resident immune cells in the brain (FIG. 25A-FIG. 25E).

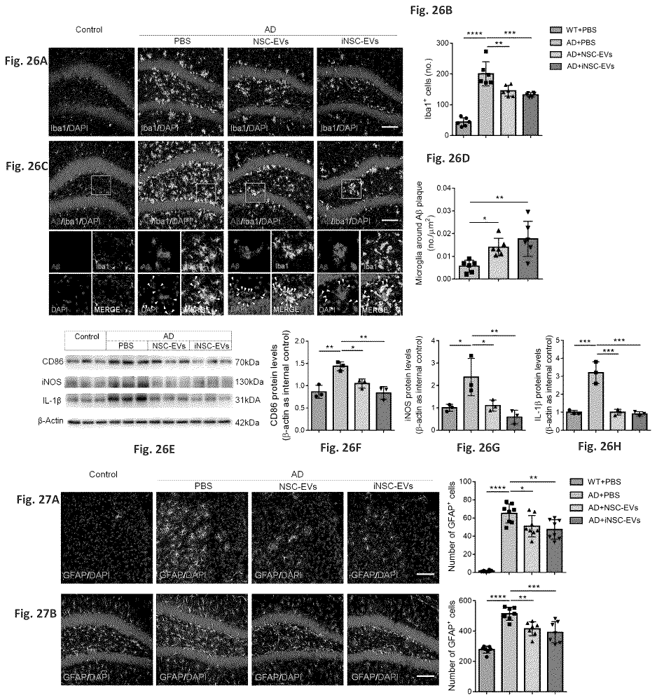

Moreover, EVs administration significantly reduced the numbers of Iba1⁺ cells and the degree of Iba1⁺ cell clustering around plaques in the PFC and hippocampus of 5×FAD mice (FIG. 33S-FIG. 33V, FIG. 26A-FIG. 26D). Western blotting results also indicated significant decline of the expression levels of pro-inflammatory proteins CD86, iNOS, and IL-1β in the PFC (FIG. 33W, FIG. 33X) and hippocampus (FIG. 26E-FIG. 26H) of EV-injected mice versus PBS controls.

In addition, astrocyte activation was also repressed by EVs, ascertained by reduced proportions of GFAP⁺ cells in both PFC and hippocampus of EV-injected 5×FAD mice, compared with PBS-injected ones (FIG. 27A-FIG. 27B).

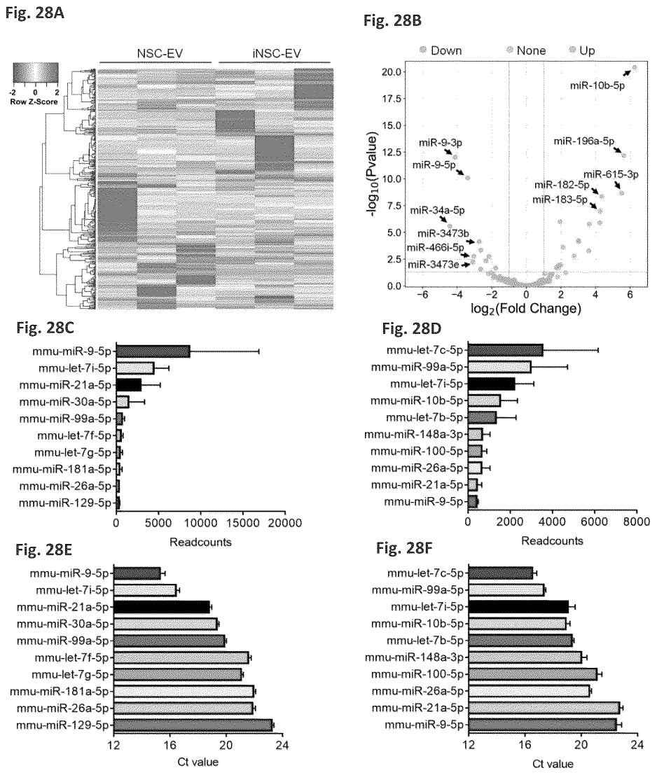

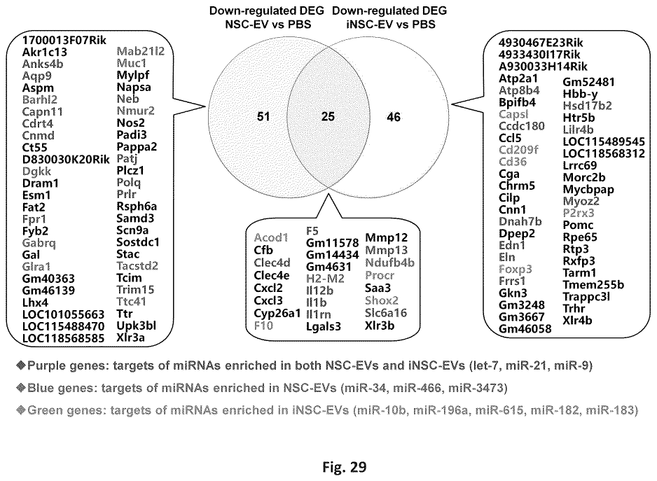

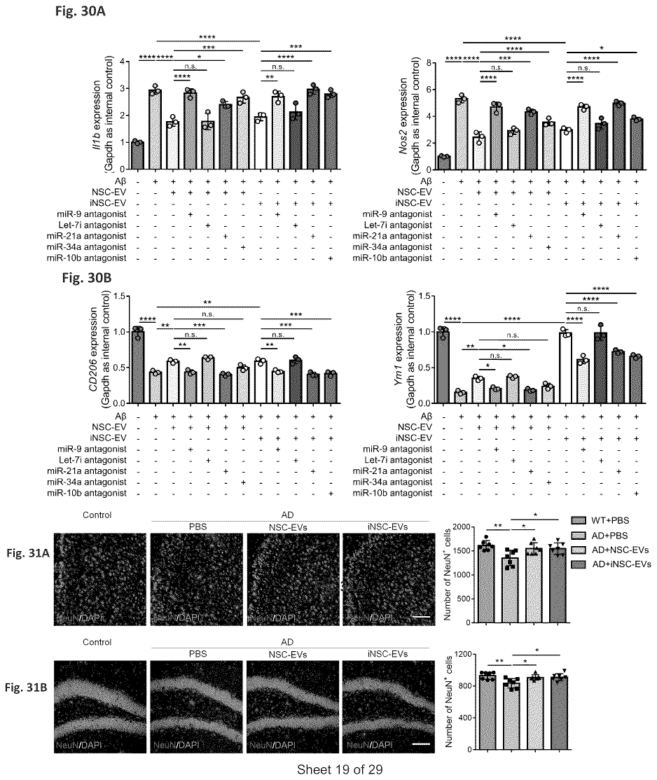

To elucidate the potential mechanisms of EV-mediated immunomodulation, the profiles of miRNAs, an important and enriched contents of EVs, in NSC-EVs and iNSC-EVs, were analyzed by microarray (FIG. 28A). Microarray results indicated the enrichment of several miRNA families (e.g., let-7, miR-9, and miR-21) in both NSC-EVs and iNSC-EVs (FIG. 28B-FIG. 28D), that are corroborated by qRT-PCR analyses (FIG. 28E, FIG. 28F). Volcano plot further identified differentially expressed miRNAs in NSC-EVs (e.g., miR-34a, etc.) versus iNSC-EVs (e.g., miR-10b, etc.) (FIG. 28B). Interestingly, around 40% of RNA-seq-identified down-regulated genes in EV-injected groups, including pro-inflammatory genes (e.g., Il1b, Il12b) and Aβ production-related ones (e.g., Mmp13, Atp8b4), are predicted to be direct targets of EV-enriched miRNAs (FIG. 29). Knockdown of EV-enriched miRNAs including let-7i, miR-21a, and miR-10b significantly reduced inhibitory effects of EVs on Aβ-induced microglial activation (FIG. 30A-FIG. 30B), implicating miRNAs as key mediators of EV-dependent immunomodulation.

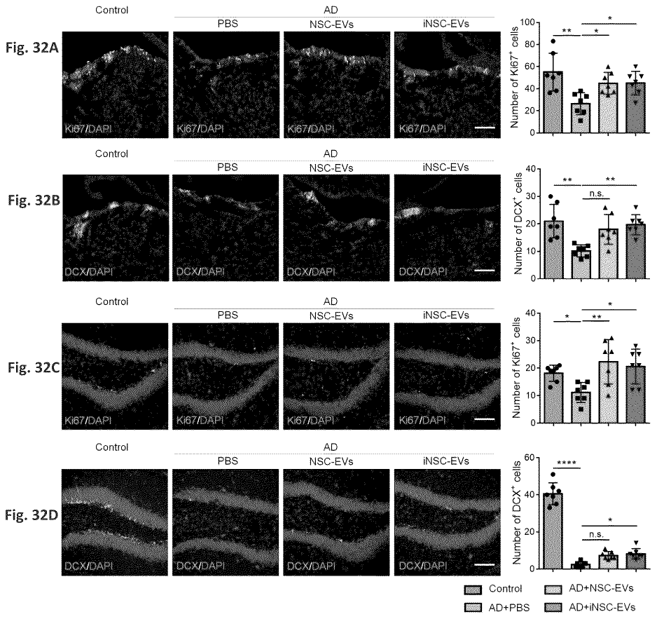

Besides, immunohistochemistry results demonstrated a significant increase in the numbers of NeuN⁺ neurons (FIG. 31A-FIG. 31B), Ki67⁺ proliferating cells, and DCX⁺ neuronal precursors (FIG. 32A-FIG. 32D) in the PFC and hippocampus of EV-injected mice versus that of PBS controls, indicating comparable effects of iNSC-EVs and NSC-EVs on restoring neuroinflammation- and Aβ deposition-induced neuronal damage and neurogenesis impairment. Thus, these results suggest that anti-neuroinflammation be an important mechanism for EV-mediated amelioration of AD-like phenotypes.

In summary, these data for the first time demonstrated that, after intravenous injection, iNSC-EVs exhibited comparable therapeutic effects with NSC-EVs on cognitive function, Aβ deposition, neuroinflammation, and neuroregeneration of 5×FAD mice, suggesting iNSC-EVs as a promising succedaneum of NSC-EVs in AD treatment.

### Example 2

This example demonstrates using extracellular vesicles derived from iNSCs to treat a patient suffering from Alzheimer's disease.

A human patient is tested to have Alzheimer's disease.

A composition comprising a therapeutically effective amount of extracellular vesicles derived from iNSCs is administered to the patient. The effectiveness and safety of the composition are tested in in vitro cell culture and in vivo animal models before being used to treat the human patient.

### Example 3

### Materials and Methods

### Cell Culture

Human neuroblastoma cells (SH-SY5Y) were grown in 1:1 EMEM/F12 medium supplemented with heat-inactivated 15% fetal bovine serum (FBS), 100 U/mL penicillin/streptomycin, 1× Non-Essential Amino Acid, and 2 mM glutamine at 37 °C in 5% CO₂ at 37 °C in a humidified tissue culture incubator.

### CCK8 Assay

Briefly, 5,000 SH-SY5Ycells were cultured on each well of 96-well plates. SH-SY5Y cells were then treated with 15 μg/ml EVs for 24 h. Cell viability was measured by CCK-8 (Yeasen, #40203ES80) assays. Experiments were handled according to the manufacturer's instructions. Absorbance was measured at 450 nm and analyzed using SpectraMax M5 microplate readers (Molecular Devices).

### Dual-Luciferase Reporter Assay

The APP/ACSL4 wild-type (wt) 3′UTR and mutated (mut) 3′UTR were synthesized by Genewiz (Genewiz, Suzhou, China) and cloned into the PmeI and SacI site of the pmirGLO vector (Promega, Beijing, China), downstream of the firefly luciferase gene. For the luciferase assay, 3,000 293T cells were cultured in 96-well plates with DMEM, 10% FBS, 100 μg/ml streptomycin, and 100 U/ml penicillin. After the confluency reached ~70%, the cells were co-transfected with the miR-26a-5p mimics and either the APP/ACSL4 3′UTR wt or 3′UTR mut dual-luciferase reporter vector. Serum-free Opti-MEM was used to prepare the transfection solution, and Lipofectamine 2000 reagent (Invitrogen) was used to facilitate the transfection according to the manufacturer's instruction. At 24 h post-transfection, Dual-Luciferase© Reporter Assay System (Promega Corporation, Beijing, China) was used to determine the luciferase activities on SpectraMax M5 microplate readers (Molecular Devices). The activity of Renilla luciferase was used to normalize that of firefly luciferase.

### DHE-Based ROS Measurement Assay

ROS production was detected using an ROS-sensitive fluorescent indicator Fluorescent Probe-Dihydroethidium (DHE) assay kit (Shanghai BestBio Biotechnology Co., Ltd). ROS generation was represented by the total DHE fluorescence, and fluorescence intensity was detected with a fluorescence microscope.

Quantitative Real-Time Polymerase Chain Reaction (qRT-PCR)

The mRNAs and miRNAs were isolated from brain tissues and cells using RNeasy mini kit (Qiagen) according to the manufacturer's instructions. Genomic DNA was removed using DNase I digestion kit (Qiagen). cDNA was synthesized using miScript II RT kit (Qiagen, Valencia, CA). Transcripts were amplified using SYBR green PCR kit (Qiagen, Valencia, CA) with the AB17500 (Applied Biosystems, Waltham, MA). All qPCR results measured each sample in triplicate, and no-template blanks were used for negative controls. Amplification curves and gene expressions were normalized to the house-keeping gene GAPDH.

### Agonist/Antagonist/siRNA and Transfection

The antagomiR control and antagomiR-26a were purchased from GenePharma (GenePharma). Transfection of 20 nM antagomiR-26a or its corresponding controls was performed using the Lipofectamine 2000 reagent (Invitrogen) according to the manufacturer's instruction.

### Results

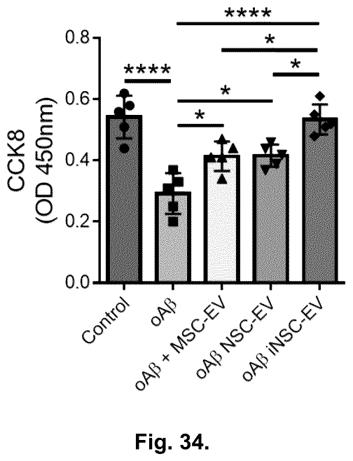

FIG. 34 shows that iNSC-EVs mitigate Aβ oligomer-induced cell damage. Human neuronal cell line SH-SY5Y cells were stimulated by Aβ oligomer, followed by EV treatment. The cell viability was determined by CCK8 assay. Error bars denote s.d. * and **** denote p<0.05 and p<0.0001, respectively. The statistical difference among groups was assessed with the parametric one-way ANOVA with post-hoc Bonferroni test.

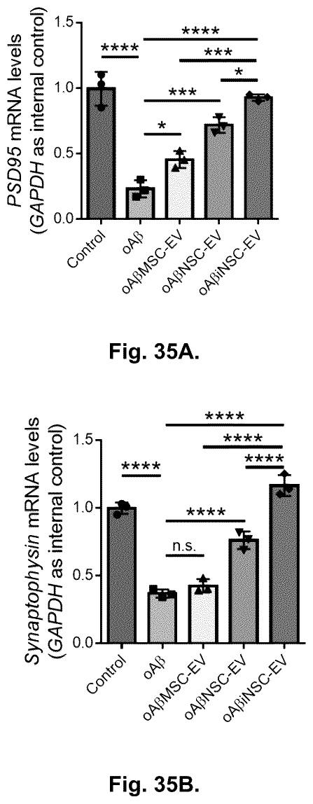

FIGS. 35A-35B show that iNSC-EVs abrogate Aβ oligomer-induced synaptic factors expression inhibition. Human neuronal cell line SH-SY5Y cells were stimulated by Aβ oligomer, followed by EV treatment. The transcript levels of post-synaptic gene PSD95 and pre-synaptic gene Synaptophysin in SH-SY5Y cells were determined by qRT-PCR. Error bars denote s.d. n.s. denotes no significance. *, ***, and **** denote p<0.05, p<0.001, and p<0.0001, respectively. The statistical difference among groups was assessed with the parametric one-way ANOVA with post-hoc Bonferroni test.

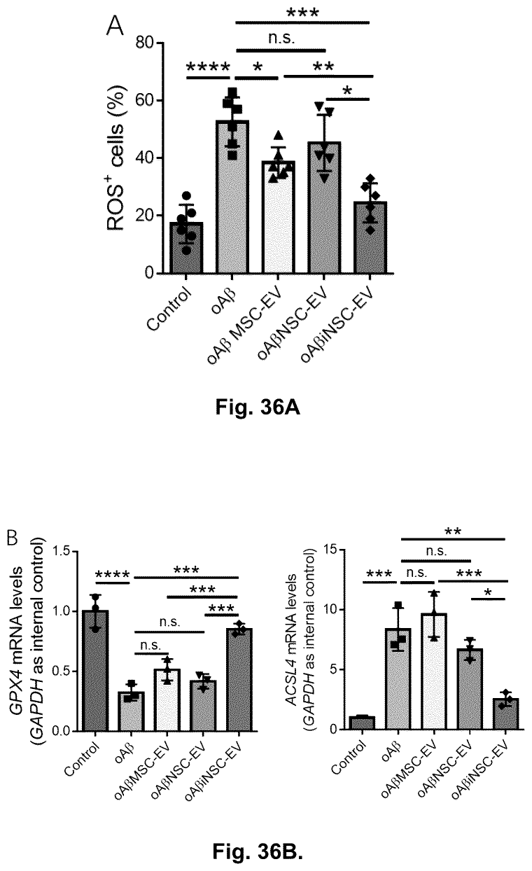

FIGS. 36A-36B show that iNSC-EVs reverse Aβ oligomer-induced oxidative stress and ferroptosis. Human neuronal cell line SH-SY5Y cells were stimulated by Aβ oligomer, followed by EV treatment. (A) ROS accumulation in SH-SY5Y cells was determined by quantification of ROS+ cells (DHE+ cells). (B) The transcript levels of anti-ferroptotic gene GPX4 and pro-ferroptotic gene ACSL4 in SH-SY5Y cells were determined by qRT-PCR. Error bars denote s.d. n.s. denotes no significance.*,**,***, and **** denote p<0.05, p<0.01, p<0.001, and p<0.0001, respectively. The statistical difference among groups was assessed with the parametric one-way ANOVA with post-hoc Bonferroni test.

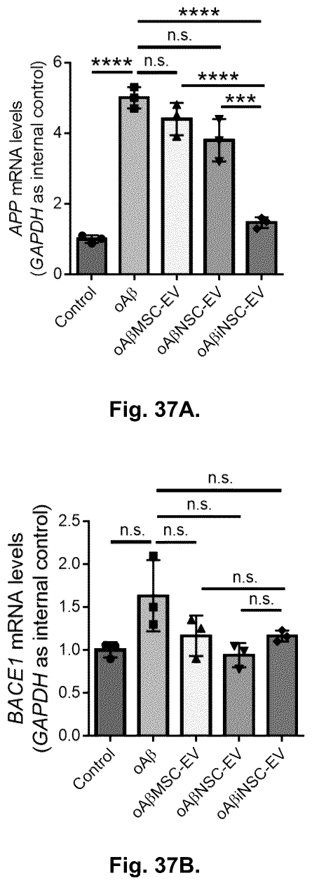

FIGS. 37A-37B show that iNSC-EVs inhibit Aβ processing genes. Human neuronal cell line SH-SY5Y cells were stimulated by Aβ oligomer, followed by EV treatment. The transcript levels of key Aβ processing genes APP and BACE1 in SH-SY5Y cells were determined by qRT-PCR. Error bars denote s.d. n.s. denotes no significance. *** and **** denote p<0.001 and p<0.0001, respectively. The statistical difference among groups was assessed with the parametric one-way ANOVA with post-hoc Bonferroni test.

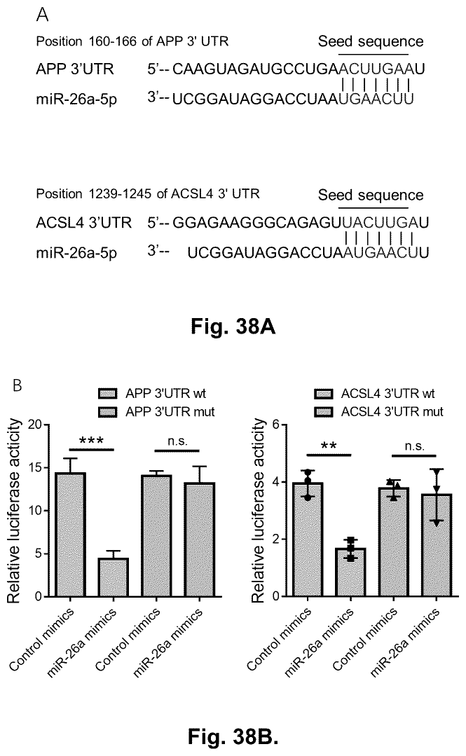

FIGS. 38A-38B show that miR-26a-5p targets the 3′UTR of APP and ACSL4 transcripts. FIG. 38A The predicted consequential pairing of 3′UTR of candidate genes and miR-26a-5p on the TargetScan website. FIG. 38B Repression of luciferase activities by the APP and ACSL4 3′UTR were dependent on miR-26a-5p. Firefly luciferase activities were normalized to the internal control, Renilla luciferase activities. Error bars denote s.d. n.s. denotes no significance. ** and *** denote p<0.01 and p<0.001, respectively. The statistical difference among groups was assessed with the parametric one-way ANOVA with post-hoc Bonferroni test.

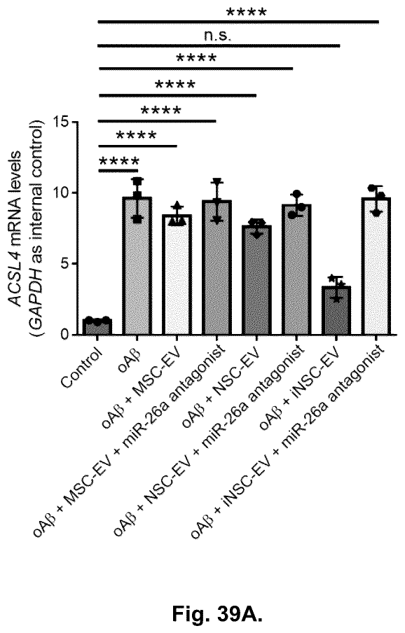

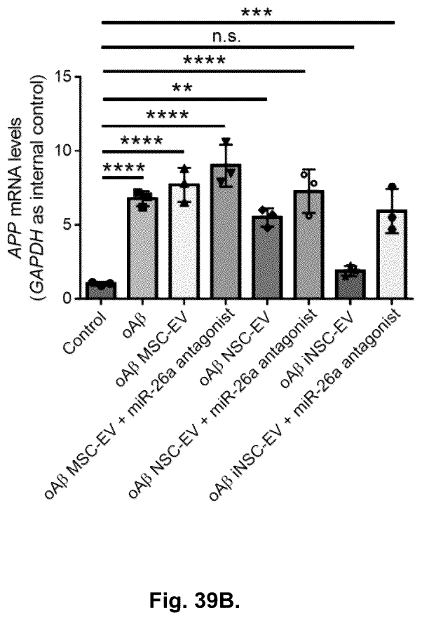

FIG. 39A-39B show that miR-26a-5p targets the 3′UTR of APP and ACSL4 transcripts. Human neuronal cell line SH-SY5Y cells were stimulated by Aβ oligomer, followed by the treatment of EVs with/without miR-26a-5p. The transcript levels of APP and ACSL4 in SH-SY5Y cells were determined by qRT-PCR. Error bars denote s.d. n.s. denotes no significance. **, ***, and **** denote p<0.01, p<0.001, and p<0.0001, respectively. The statistical difference among groups was assessed with the parametric one-way ANOVA with post-hoc Bonferroni test.

### REFERENCES

1. G. M. McKhann, D. S. Knopman, H. Chertkow, B. T. Hyman, C. R. Jack Jr., C. H. Kawas, W. E. Klunk, W. J. Koroshetz, J. J. Manly, R. Mayeux, et al. The diagnosis of dementia due to Alzheimer's disease: recommendations from the National Institute on Aging-Alzheimer's Association workgroups on diagnostic guidelines for Alzheimer's disease. Alzheimers Dement., 7 (2011), pp. 263-269.
2. L. Vermunt, S. A. M. Sikkes, A. van den Hout, R. Handels, I. Bos, W. M. van der Flier, S. Kern, P.-J. Ousset, P. Maruff, I. Skoog, et al. Duration of preclinical, prodromal, and dementia stages of Alzheimer's disease in relation to age, sex, and APOE genotype. Alzheimers Dement., 15 (2019), pp. 888-898.
3. Leng, F., Edison, P. Neuroinflammation and microglial activation in Alzheimer disease: where do we go from here? Nat Rev Neurol. 17(3):157-172 (2021).
4. Xia, X., Wang, Y., Zheng, J. C. Extracellular vesicles, from the pathogenesis to the therapy of neurodegenerative diseases. Transl Neurodegener. 11(1):53 (2022).
5. Ma, Y. et al. Exosomes released from neural progenitor cells and induced neural progenitor cells regulate neurogenesis through miR-21a. Cell Commun Signal. 17(1):96 (2019).
6. Gao, G. et al. Induced neural stem/progenitor cell-derived extracellular vesicles promote recovery post-stroke. Clin Transl Med. 12(6):e936 (2022).

1. A method for treating Alzheimer's disease in a patient in need thereof, comprising administration to the patient of a composition comprising a therapeutically effective amount of extracellular vesicles.

2. The method of claim 1, wherein the extracellular vesicles are isolated from culture medium of cells.

3. The method of claim 1, wherein the cells are neural stem cells (NSCs), mesenchymal stem cells (MSCs), or fibroblast derived induced NSCs (iNSCs).

4. The method of claim 1, wherein the extracellular vesicles comprise one or more of following miRNAs: let-7, miR-9, miR-21, miR-34a, and miR-10b.

5. (canceled)

6. The method of claim 1, wherein the administration is at least once a day or more times a day, or at least once in three days, or at least for a month.

7. (canceled)

8. (canceled)

9. The method of claim 1, wherein the administration is via oral, intravenous injectable, intramuscular injectable, intraperitoneal, intranasal, rectal, or sublingual route.

10. The method of claim 1, wherein the administration of the composition is by a dose in the range of 2-2,000 mg, 2-500 mg, 2-200 mg, 2-150 mg, 5-100 mg, or 5-50 mg, or wherein the dose is administered in divided doses or a single dose.

11. (canceled)

12. The method of claim 1, wherein the composition down-regulates levels of Aβ₁₋₄₂ in prefrontal cortex (PFC) and hippocampus.

13. The method of claim 1, wherein the composition reduces phosphorylated Tau (pTau) propagation.

14. The method of claim 1, wherein the composition regulates neuroinflammation.

15. The method of claim 1, wherein the composition reduces levels of CD86, iNOS, and IL-1β in the PFC and hippocampus.

16. The method of claim 1, wherein the composition reduces one or more of following gene expression: Il1b, Il12b, Mmp13, and Atp8b4.

17. A composition comprising a therapeutically effective amount of extracellular vesicles for treating Alzheimer's disease in a patient in need thereof, wherein the extracellular vesicles are isolated from culture medium of cells.

18. (canceled)

19. The composition of claim 17, wherein the cells are neural stem cells (NSCs), mesenchymal stem cells (MSCs), or fibroblast derived induced NSCs (iNSCs).

20. The composition of claim 17, wherein the extracellular vesicles comprise one or more of following miRNAs: let-7, miR-9, miR-21, miR-34a, and miR-10b.

21. (canceled)

22. The composition of claim 17, wherein the composition down-regulates levels of Aβ₁₋₄₂ in prefrontal cortex (PFC) and hippocampus.

23. The composition of claim 17, wherein the composition reduces phosphorylated Tau (pTau) propagation.

24. The composition of claim 17, wherein the composition regulates neuroinflammation.

25. The composition of claim 17, wherein the composition reduces levels of CD86, iNOS, and IL-1β in the PFC and hippocampus.

26. The composition of claim 17, wherein the composition reduces one or more of following gene expression: Il1b, Il12b, Mmp13, and Atp8b4.

27. (canceled)

28. (canceled)

29. (canceled)

30. (canceled)

31. (canceled)


SEQUENCE LISTING (31 sequences)

SEQ ID NO: 1 - DNA, 23 bp, synthetic construct
  source 1..23: mol_type = other DNA
catgttccagtatgactccactc

SEQ ID NO: 2 - DNA, 20 bp, synthetic construct
  source 1..20: mol_type = other DNA
ggcctcaccccatttgatgt

SEQ ID NO: 3 - DNA, 23 bp, synthetic construct
  source 1..23: mol_type = other DNA
ccagcaggttatcatcatcatcc

SEQ ID NO: 4 - DNA, 19 bp, synthetic construct
  source 1..19: mol_type = other DNA
ctcgcagcagcacatcaac

SEQ ID NO: 5 - DNA, 22 bp, synthetic construct
  source 1..22: mol_type = other DNA
cccttcaatggttggtacatgg

SEQ ID NO: 6 - DNA, 21 bp, synthetic construct
  source 1..21: mol_type = other DNA
acattgatctccgtgacagcc

SEQ ID NO: 7 - DNA, 20 bp, synthetic construct
  source 1..20: mol_type = other DNA
acgtggaactggcagaagag

SEQ ID NO: 8 - DNA, 20 bp, synthetic construct
  source 1..20: mol_type = other DNA
ggtctgggccatagaactga

SEQ ID NO: 9 - DNA, 20 bp, synthetic construct
  source 1..20: mol_type = other DNA
cgggtccctggactccttca

SEQ ID NO: 10 - DNA, 20 bp, synthetic construct
  source 1..20: mol_type = other DNA
cgcgtttccagctctcggat

SEQ ID NO: 11 - DNA, 23 bp, synthetic construct
  source 1..23: mol_type = other DNA
tcacaggtctggcaattcttctg

SEQ ID NO: 12 - DNA, 22 bp, synthetic construct
  source 1..22: mol_type = other DNA
actcccttctattggcctgtcc

SEQ ID NO: 13 - DNA, 21 bp, synthetic construct
  source 1..21: mol_type = other DNA
tctttgcctttcccagtctcc

SEQ ID NO: 14 - DNA, 19 bp, synthetic construct
  source 1..19: mol_type = other DNA
tgacacccagcggaatttc

SEQ ID NO: 15 - DNA, 20 bp, synthetic construct
  source 1..20: mol_type = other DNA
gaatcgagcaccagttacgc

SEQ ID NO: 16 - DNA, 19 bp, synthetic construct
  source 1..19: mol_type = other DNA
tggcccctgcgcaaggatg

SEQ ID NO: 17 - DNA, 23 bp, synthetic construct
  source 1..23: mol_type = other DNA
tctttggttatctagctgtatga

SEQ ID NO: 18 - DNA, 22 bp, synthetic construct
  source 1..22: mol_type = other DNA
tgaggtagtagtttgtgctgtt

SEQ ID NO: 19 - DNA, 23 bp, synthetic construct
  source 1..23: mol_type = other DNA
taccctgtagaaccgaatttgtg

SEQ ID NO: 20 - DNA, 22 bp, synthetic construct
  source 1..22: mol_type = other DNA
tagcttatcagactgatgttga

SEQ ID NO: 21 - DNA, 22 bp, synthetic construct
  source 1..22: mol_type = other DNA
tgtaaacatcctcgactggaag

SEQ ID NO: 22 - DNA, 22 bp, synthetic construct
  source 1..22: mol_type = other DNA
aacccgtagatccgatcttgtg

SEQ ID NO: 23 - DNA, 22 bp, synthetic construct
  source 1..22: mol_type = other DNA
tgaggtagtagattgtatagtt

SEQ ID NO: 24 - DNA, 22 bp, synthetic construct
  source 1..22: mol_type = other DNA
tgaggtagtagtttgtacagtt

SEQ ID NO: 25 - DNA, 23 bp, synthetic construct
  source 1..23: mol_type = other DNA
aacattcaacgctgtcggtgagt

SEQ ID NO: 26 - DNA, 22 bp, synthetic construct
  source 1..22: mol_type = other DNA
ttcaagtaatccaggataggct

SEQ ID NO: 27 - DNA, 21 bp, synthetic construct
  source 1..21: mol_type = other DNA
ctttttgcggtctgggcttgc

SEQ ID NO: 28 - DNA, 22 bp, synthetic construct
  source 1..22: mol_type = other DNA
tgaggtagtaggttgtatggtt

SEQ ID NO: 29 - DNA, 22 bp, synthetic construct
  source 1..22: mol_type = other DNA
tgaggtagtaggttgtgtggtt

SEQ ID NO: 30 - DNA, 22 bp, synthetic construct
  source 1..22: mol_type = other DNA
tcagtgcactacagaactttgt

SEQ ID NO: 31 - DNA, 22 bp, synthetic construct
  source 1..22: mol_type = other DNA
aacccgtagatccgaacttgtg